(19) United States
(12) Patent Application Publication
(10) Pub. No.: US 2025/0268580 A1
(43) Pub. Date: Aug. 28, 2025

(54) WIRED / WIRELESS MULTIPLE-MODALITY SYSTEMS FOR MULTIPLE CATHETERS IN INTERVENTIONAL CARDIOLOGY

(71) Applicant: yoR Labs, Inc., Beaverton, OR (US)

(72) Inventors: Anshumali Roy, Portland, OR (US); Glen Wallace McLaughlin, Woodside, CA (US); Dakota Pellegrino, Hillsboro, OR (US); Nikki Troiano Gainey, Apex, NC (US)

(21) Appl. No.: 19/092,438

(22) Filed: Mar. 27, 2025

Related U.S. Application Data
(63) Continuation of application No. 19/109,041, filed on Jan. 1, 0001.
(60) Provisional application No. 63/375,206, filed on Sep. 9, 2022.

(51) Int. Cl. A61B 8/00; A61B 8/12; A61B 17/00; A61B 18/00; A61B 18/14; G06T 7/00

(52) U.S. Cl. CPC A61B 8/5269; A61B 8/12; A61B 8/5207; A61B 18/1492; G06T 7/0012; A61B 8/4461; A61B 8/54; A61B 2017/00106; A61B 2018/00904; G06T 2207/10132; G06T 2207/20084; G06T 2207/30096

(57) ABSTRACT

A multiple-modality catheter system disposed in a housing comprises a battery pack, a wireless module to communicate with a control console, a remote control or remote controls and a display or displays, a computing module, a modality circuitry module to control a plurality of catheter modules, and a connector or a plurality of connectors adapted to connect to a plurality of catheters. The multiple-modality catheter system may be highly integrated to fit a reusable universal catheter handle. The multiple-modality system includes a synchronization circuitry to minimize interference between the catheter modalities to get high quality ultrasound images. The ultrasound image quality improvement feature can be further enhanced by system noise detection and correction. And the multiple-modality system disclosed can be used for lesion tracking and ablation by applying deep-learning.

### INCORPORATION BY REFERENCE TO ANY PRIORITY APPLICATIONS

Any and all application for which a foreign or domestic priority claim is identified in the Application Data Sheet as filed with the present application are hereby incorporated by reference under 37 CFR 1.57. This disclosure is a continuation of U.S. application Ser. No. 19/109,041, filed on Mar. 5, 2025, which is a national phase filing of PCT/US2023/073675, filed on Sep. 7, 2023, which claims priority to U.S. Provisional No. 63/375,206, filed on Sep. 9, 2022, all of which are incorporated by reference herein in their entirety.

### TECHNICAL FIELD

The invention relates to catheter medical systems. Specifically, the invention relates to catheter devices for cardiac or vascular diagnostic and interventional procedures.

### BACKGROUND

Catheters are commonly used to access patient anatomy for medical reasons. Catheters are often exposed to biomaterials including bodily fluids, tissue, and pathogens. Accordingly, catheters are typically single-use devices to be disposed of after each use to prevent the transmission of biomaterials from one patient to another. Due to their single-use nature and to minimize costs, catheters are often constructed from inexpensive materials with simple mechanical elements for providing controls and lack sophisticated electrical components and control systems.

Instead of processing data itself, catheters transmit data, including imaging data, to separate sterilized equipment. The data is often complex and includes large data sets requiring the use of cables extending from the catheter to the data processing equipment. Some equipment is only configured to process certain types of data. Accordingly, operating rooms may comprise a plurality of processing equipment configured to process a certain category of data and a plurality of cables extending from the equipment to the catheters. Additionally, operating rooms are often small and have many care takers present during an operation. Thus, the plurality of cables presents a hazard within the operating rooms. Thus, there remains a need to simplify the operating room by removing physical tripping hazards and minimizing the number of unnecessary or redundant equipment.

### SUMMARY

Certain aspects of this invention are defined by the independent claims. The dependent claims concern optional features of some embodiments of the invention. The systems, methods, and devices described herein each have several aspects, no single one of which is solely responsible for its desirable attributes. Without limiting the scope of this disclosure, several non-limiting features will now be discussed briefly.

One innovation includes a multiple-modality system disposed in a housing. The multiple-modality system includes a battery module, a wireless module capable of transmitting wireless signals to communicate with peripheral equipment, a computing module having electronics and software residing thereon capable of processing image and data acquired from one or more catheters or probes, and a modality circuitry module capable of supporting a plurality of catheter or probe modalities.

In another innovation, the modality circuitry module in the multiple-modality system can support an intracardiac echocardiography (ICE) catheter modality and an intravascular ultrasound (IVUS) catheter modality.

In another innovation, the modality circuitry module in the multiple-modality system can support an intracardiac echocardiography (ICE) catheter modality, an intravascular ultrasound (IVUS) catheter modality, and a radiofrequency (RF) ablation catheter modality.

In another innovation, the modality circuitry module in the multiple-modality system can support an intracardiac echocardiography (ICE) catheter modality, an intravascular ultrasound (IVUS) catheter modality, a radiofrequency (RF) ablation catheter modality, and a fractional flow reserve (FFR) catheter modality.

In another innovation, the modality circuitry module in the multiple-modality system may support any two or more of the following catheters: ICE, IVUS, RF ablation, TEE and an externally applied TTE probe.

In another innovation, the housing of the multiple-modality system is a chassis, and the chassis is equal to or smaller than 12 inch×10 inch×4 inch in size.

In another innovation, the housing of the multiple-modality system is a universal catheter handle that can support a plurality of modality systems.

Another innovation includes a synchronization system capable of reducing interference between a plurality of modalities for high ultrasound image quality or modality performance. The synchronization system comprises a scheduler capable of sending out signals to cause sequential operation of the plurality of modalities forming repetitive cycles with a cycling frequency, wherein at a specific time no more than one modality is running, and a controller to start, pause, and stop each of the plurality of modalities according to signals received from the scheduler.

In another innovation, the controller of the synchronization system includes clock to generate pulse signal with a clock pulse frequency, wherein the clock pulse frequency is substantially higher than the cycling frequency, a clock-gating controller capable of starting, pausing, or stopping the clocking of each of the plurality of modalities, a power-gating controller capable of controlling momentary, longer, or shutting down each of the plurality of modalities.

In another innovation, the synchronization system can synchronize an intracardiac echocardiography (ICE) catheter modality and an intravascular ultrasound (IVUS) catheter modality and get high quality ultrasound images.

In another innovation, the synchronization system can synchronize an intracardiac echocardiography (ICE) catheter modality and a radiofrequency (RF) ablation catheter modality and get high quality ICE images and optimal RF ablation performance.

In another innovation, the synchronization system can synchronize an intracardiac echocardiography (ICE) catheter modality, a radiofrequency (RF) ablation catheter modality, and a fractional reserve (FFR) catheter modality and get optimal performance for each catheter modality.

In another innovation, the synchronization system can synchronize an intracardiac echocardiography (ICE) catheter modality, a transthoracic echocardiography (TTE) probe modality, and a transesophageal echocardiography (TEE) catheter modality and get optimal performance for each modality.

In another innovation, the synchronization system can synchronize an intracardiac echocardiography (ICE) catheter modality, a transthoracic echocardiography (TTE) probe modality, a transesophageal echocardiography (TEE) catheter modality, and a radiofrequency (RF) ablation catheter modality and get optimal performance for each modality.

In another innovation, the synchronization system can synchronize any combinations of two or more of the following catheters or probes: ICE, IVUS, RF Ablation, TEE, and TTE (e.g., an externally applied TTE probe).

In another innovation, a multiple-modality system disposed in a housing. comprises a battery module, and a communication module, the communication module having a transmitter capable of transmitting signals to communicate with peripheral equipment. The a multiple-modality system can further include a computing module having electronics and software loaded thereon capable of processing data, and a modality circuitry module, the modality circuitry module capable of supporting a plurality of modalities. The modalities can include, for example, any combination of ICE, IVUS, RF Ablation, TEE, and TTE functionality. For example, the multiple-modality system is configured for control and processing for any two or more of any of ICE, IVUS, RF Ablation, TEE, and TTE (e.g., an externally applied TTE probe).

In one aspect, a multiple-modality system disposed in a housing comprises a battery module; a wireless module, the wireless module capable of transmitting wireless signals to communicate with peripheral equipment; a computing module, the computing module having electronics and software loaded thereon capable of processing data; and a modality circuitry module, the modality circuitry module capable of supporting a plurality of modalities. In some aspects, the modality circuitry module is capable of supporting an intracardiac echocardiography (ICE) catheter modality and an intravascular ultrasound (IVUS) catheter modality. In some aspects, the modality circuitry module is capable of supporting an intracardiac echocardiography (ICE) catheter modality, an intravascular ultrasound (IVUS) catheter modality, and a radiofrequency (RF) ablation catheter modality. In some aspects, the modality circuitry module is capable of supporting an intracardiac echocardiography (ICE) catheter modality, an intravascular ultrasound (IVUS) catheter modality, a radiofrequency (RF) ablation catheter modality, and a fractional flow reserve (FFR) catheter modality. In some aspects, the modality circuitry module is capable of supporting an intracardiac echocardiography (ICE) catheter modality, a transthoracic echocardiography (TTE) probe modality, and a transesophageal echocardiography (TEE) catheter modality. In some aspects, the modality circuitry module is capable of supporting modalities selected from the group of modalities consisting of an intracardiac echocardiography (ICE) catheter modality, a transthoracic echocardiography (TTE) probe modality, a transesophageal echocardiography (TEE) catheter modality, a radiofrequency (RF) ablation catheter modality. In some aspects, the housing is a chassis with dimensions equal to or smaller than 15 inch×12 inch×6 inch. In some aspects, the dimensions of the chassis is equal to or smaller than 12 inch×10 inch×4 inch. In some aspects, the housing is a reusable catheter handle. In some aspects, the reusable catheter handle is a universal catheter handle capable of supporting a plurality of catheter modalities. In some aspects, the battery module includes a rechargeable battery set. In some aspects, when fully charged the rechargeable battery set is capable of suppling power to operate the multiple-modality system for more than 4 hours.

In one aspect, a synchronization system capable of reducing interference for a plurality of modality systems, comprises a scheduler, the scheduler capable of sending out signals to cause the plurality of modalities to operate sequentially forming repetitive operation cycles with a cycling frequency, wherein at a specific time no more than one modality is running; and a controller, the controller starting and stopping each of the plurality of modalities according to signals received from the scheduler. In some aspects, the controller includes a clock synchronizer, the clock synchronizer capable of generating a clock pulse signal with a clock pulse frequency, wherein the clock pulse frequency is substantially higher than the cycling frequency; a clock-gating controller, the clock-gating controller capable of starting, pausing, and stopping the clocking for each of the plurality of modalities according to the signals from the scheduler; a power-gating controller, the power-gating controller capable of starting, pausing, and stopping the power for each of the plurality of modalities according to the signals from the scheduler; and wherein each of the plurality of modalities is in operation mode only when the clocking is started by the clock-gating controller for the modality and the power is started by the power-gating controller. In some aspects, each of the plurality of modality systems includes an intracardiac echocardiography (ICE) catheter modality and an intravascular ultrasound (IVUS) catheter modality. In some aspects, each of the plurality of modality systems includes an intracardiac echocardiography (ICE) catheter modality, an intravascular ultrasound (IVUS) catheter modality, and a radiofrequency (RF) ablation catheter modality. In some aspects, each of the plurality of modality systems includes an intracardiac echocardiography (ICE) catheter modality, an intravascular ultrasound (IVUS) catheter modality, a radiofrequency (RF) ablation catheter modality, and a fractional flow reserve (FFR) catheter modality. In some aspects, each of the plurality of modality systems includes an intracardiac echocardiography (ICE) catheter modality, a transthoracic echocardiography (TTE) probe modality, and a transesophageal echocardiography (TEE) catheter modality. In some aspects, each of the plurality of modality systems includes modalities selected from the group consisting of an intracardiac echocardiography (ICE) catheter modality, a transthoracic echocardiography (TTE) probe modality, a transesophageal echocardiography (TEE) catheter modality, and a radiofrequency (RF) ablation catheter modality.

In one aspect, a method for synchronizing a plurality of modality systems for optimal performance for each of the modality systems, comprises the steps of: establishing a scheduler, the scheduler capable of sending out signals to cause the plurality of modalities to operate sequentially forming repetitive operation cycles with a cycling frequency, wherein at a specific time no more than one modality is running; and establishing a controller, the controller starting and stopping each of the plurality of modalities according to signals received from the scheduler. In some aspects, the step of establishing a controller comprising the steps of: generating a clock pulse signal with a clock pulse frequency, wherein the clock pulse frequency is substantially higher than the cycling frequency; establishing a clock-gating controller, the clock-gating controller capable of starting, pausing, and stopping the clocking for each of the plurality of modalities according to the signals from the scheduler; establishing a power-gating controller, the power-gating controller capable of starting, pausing, and stopping the power for each of the plurality of modalities according to the signals from the scheduler; and wherein each of the plurality of modalities is in operation mode only when the clocking is started by the clock-gating controller for the modality and the power is started by the power-gating controller. In some aspects, each of the plurality of modality systems includes an intracardiac echocardiography (ICE) catheter modality and an intravascular ultrasound (IVUS) catheter modality. In some aspects, each of the plurality of modality systems includes an intracardiac echocardiography (ICE) catheter modality, an intravascular ultrasound (IVUS) catheter modality, and a radiofrequency (RF) ablation catheter modality. In some aspects, each of the plurality of modality systems includes an intracardiac echocardiography (ICE) catheter modality, an intravascular ultrasound (IVUS) catheter modality, a radiofrequency (RF) ablation catheter modality, and a fractional flow reserve (FFR) catheter modality. In some aspects, each of the plurality of modality systems includes an intracardiac echocardiography (ICE) catheter modality, a transthoracic echocardiography (TTE) probe modality, and a transesophageal echocardiography (TEE) catheter modality. In some aspects, each of the plurality of modality systems includes modalities selected from the group consisting of an intracardiac echocardiography (ICE) catheter modality, a transthoracic echocardiography (TTE) probe modality, a transesophageal echocardiography (TEE) catheter modality, and a radiofrequency (RF) ablation catheter modality.

In one aspect, a method for detecting and ablating lesion using a multiple-modality system including an intracardiac echocardiography (ICE) catheter and a radiofrequency (RF) ablation catheter, comprising the steps of: generating an ultrasound image frame with the ICE catheter; determining if lesion is detected using a lesion detection convolutional neural network (CNN); if lesion is detected, optimizing ablation power and pulse for the RF ablation catheter and proceeding to ablate the lesion; and if lesion is not detected, going back to the beginning of the loop to generate another ultrasound image frame with the ICE catheter. In some aspects, the method further comprises the steps of: if lesion is detected, determining if deep-learning guided lesion imaging is needed; if the answer yes, rotating the ICE catheter tip using servo control and proceeding to determine if full lesion ablation is acquired; and if the answer is yes, going to the step of optimizing ablation power and pulse for the RF ablation catheter and proceeding to ablate the lesion.

In one aspect, a method for detecting and correcting noise in a multiple-modality system including an echocardiography modality for optimal ultrasound image quality, comprises the steps of: generating an ultrasound image frame using the echocardiography modality; detecting noise using a noise detector; determining if abnormal ultrasound noise is detected; if noise is detected, proceeding to adjust ultrasound control parameters and go back to the beginning of the loop to generate an ultrasound image frame; and if noise is not detected, proceeding to noise frequency analysis and auto filter generation for denoising and go back to generate an ultrasound image frame. In some aspects, the echocardiography modality is an ICE catheter. In some aspects, the echocardiography modality is a TEE catheter. In some aspects, the echocardiography modality is a TTE/general probe.

In one aspect, a multiple-modality system disposed in a housing, comprises: a battery module; a communication module, the communication module capable of transmitting signals to communicate with peripheral equipment; a computing module, the computing module having electronics and software loaded thereon capable of processing data; and a modality circuitry module, the modality circuitry module capable of supporting a plurality of modalities. In some aspects, the modality circuitry module is capable of supporting an intracardiac echocardiography (ICE) catheter modality and an intravascular ultrasound (IVUS) catheter modality. In some aspects, the modality circuitry module is capable of supporting an intracardiac echocardiography (ICE) catheter modality, an intravascular ultrasound (IVUS) catheter modality, and a radiofrequency (RF) ablation catheter modality. In some aspects, the modality circuitry module is capable of supporting an intracardiac echocardiography (ICE) catheter modality, an intravascular ultrasound (IVUS) catheter modality, a radiofrequency (RF) ablation catheter modality, and a fractional flow reserve (FFR) catheter modality. In some aspects, the modality circuitry module is capable of supporting an intracardiac echocardiography (ICE) catheter modality, a transthoracic echocardiography (TTE) probe modality, and a transesophageal echocardiography (TEE) catheter modality. In some aspects, the modality circuitry module is capable of supporting modalities selected from the group of modalities consisting of an intracardiac echocardiography (ICE) catheter modality, a transthoracic echocardiography (TTE) probe modality, a transesophageal echocardiography (TEE) catheter modality, a radiofrequency (RF) ablation catheter modality. In some aspects, the housing is a chassis with dimensions equal to or smaller than 15 inch×12 inch×6 inch. In some aspects, the dimensions of the chassis is equal to or smaller than 12 inch×10 inch×4 inch. In some aspects, the housing is a reusable catheter handle. In some aspects, the reusable catheter handle is a universal catheter handle capable of supporting a plurality of catheter modalities. In some aspects, the battery module includes a rechargeable battery set. In some aspects, when fully charged the rechargeable battery set is capable of suppling power to operate the multiple-modality system for more than 4 hours.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic illustrating an example embodiment of a multiple-modality system 100 having an ICE and IVUS ultrasound circuitry module 107.

FIG. 2 is a perspective view of a universal catheter handle 135 used with the multiple-modality system 100 in FIG. 1.

FIG. 3 a perspective view of the universal catheter handle 135 of FIG. 2 viewed from a different angle.

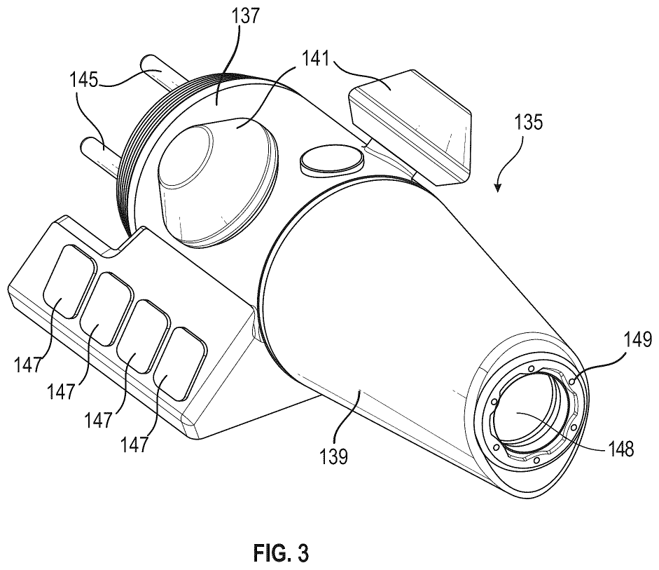

FIG. 4 is a schematic illustrating an example embodiment of a multiple-modality system 200 which is an alternative embodiment of the multiple-modality system 100 in FIG. 1.

FIG. 5 is a schematic illustrating an example embodiment of a multiple-modality system 300 having an ICE and IVUS ultrasound circuitry module 307 and an RF ablation circuitry module 308.

FIG. 6 is a schematic of synchronization control system 361 of an ICE catheter and an RF ablation catheter.

FIG. 7 is a schematic showing the components of the synchronization system 361 in FIG. 6.

FIG. 8 is a schematic illustrating the cycling between the ICE modality for ultrasound image acquisition and the RF ablation modality for the ablation operation.

FIG. 9 is a schematic illustrating the alternating ICE data acquisition period and the RF ablation period as controlled by clock gating.

FIG. 10 is a schematic illustrating power on and off scheme of both the ICE catheter and the RF catheter as controlled by power gating.

FIG. 11 is a schematic illustrating an example embodiment of a multiple-modality system 400 which is an alternative embodiment of the multiple-modality system 300 in FIG. 5.

FIG. 12 is a schematic illustrating an example embodiment of a multiple-modality system 500 having an ICE and IVUS ultrasound circuitry module 507, an RF ablation circuitry module 508, and an FFR interface circuitry module 509.

FIG. 13 is a schematic illustrating an example embodiment of a multiple-modality system 600 which is an alternative embodiment of the multiple-modality system 500 in FIG. 12.

FIG. 14 is a schematic of a synchronization control system for three ultrasound probe/catheter modalities, including an ICE catheter a TTE probe, and TEE catheter.

FIG. 15 is a schematic showing the components of the synchronization system in FIG. 14.

FIG. 16 is a schematic illustrating the cycling operations for the three-ultrasound probe/catheters.

FIG. 17 is a schematic of a synchronization control system for the ICE ultrasound catheter modality and the TEE ultrasound catheter modality.

FIG. 18 is a schematic of a synchronization control system for the ICE ultrasound catheter modality and the general/TTE ultrasound probe modality.

FIG. 19 is a schematic of a synchronization control system for the TEE ultrasound catheter modality and the general/TTE ultrasound probe modality.

FIG. 20 is a schematic of a synchronization control system of three ultrasound probe/catheter modalities, an ICE catheter, a TTE probe, and a TEE catheter, together with an RF ablation catheter.

FIG. 21 is a schematic showing the components of the synchronization system in FIG. 20.

FIG. 22 is a schematic illustrating the cycling operations for the four-ultrasound probe/catheters.

FIG. 23 is a schematic of a synchronization control system for a TEE ultrasound catheter and an RF ablation catheter.

FIG. 24 is a schematic of a synchronization control system for a general/TTE ultrasound probe and an RF ablation catheter.

FIG. 25 is a schematic of a synchronization control system for an ICE catheter, a TEE catheter, and an RF ablation catheter.

FIG. 26 is a schematic of a synchronization control system for an ICE catheter, a general/TTE probe, and an RF ablation catheter.

FIG. 27 is a schematic of a synchronization control system for a TEE catheter, a general/TTE probe, and an RF ablation catheter.

FIG. 28 is a schematic of a lesion inference layer 700 involving deep-learning working together with a synchronization control system for an ICE catheter and an RF ablation catheter.

FIG. 29 is a schematic showing the components of the lesion inference layer 700 and the synchronization system in FIG. 28.

FIG. 30 is a flowchart illustrating the deep learning process for the lesion inference layer 700 in FIG. 28.

FIG. 31 is a flowchart illustrating how the lesion inference layer 700 works in an RF ablation procedure.

FIG. 32 is a schematic showing an automated lesion inference layer 700 connected to a multiple-modality system and with an ICE catheter tip rotation control added.

FIG. 33 is a flowchart showing the steps of FIG. 31 in addition to automated servo control of ICE catheter tip rotation for full lesion acquiring.

FIG. 34 is a schematic illustrating a noise detection/correction system 800 connected to the multiple-modality system in FIG. 20 to further enhance ultrasound image quality.

FIG. 35 is a schematic illustration more detailed connection of the noise detection/correction system 800 in FIG. 34 to the synchronization circuitry and the modalities.

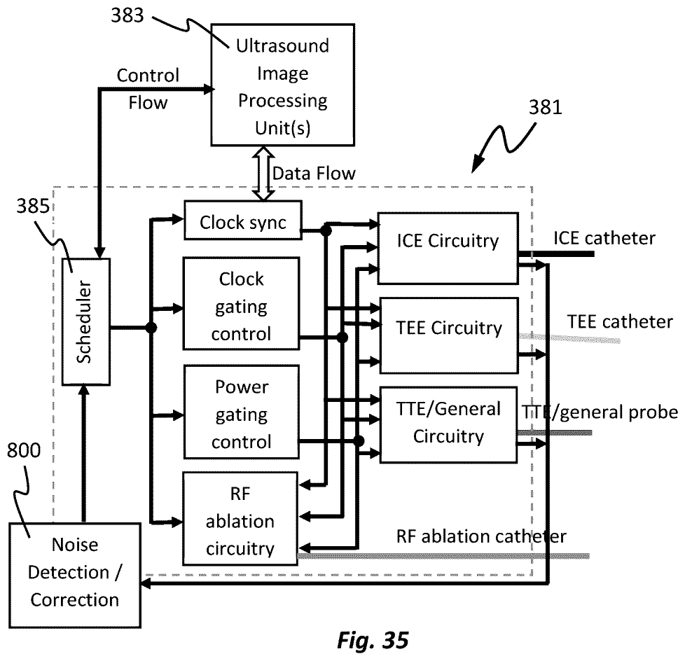

FIG. 36 is a flowchart showing how the noise detection/correction system 800 works.

FIG. 37 is a schematic showing the noise detection/correction system 800 in FIG. 34 working in a multiple-modality system including an ICE catheter and a RF ablation catheter.

FIG. 38 is a schematic showing the noise detection/correction system 800 in FIG. 34 working in a multiple-modality system including a TEE catheter and a RF ablation catheter.

FIG. 39 is a schematic showing the noise detection/correction system 800 in FIG. 34 working in a multiple-modality system including a general/TTE probe and a RF ablation catheter.

FIG. 40 is a schematic showing the noise detection/correction system 800 in FIG. 34 working in a multiple-modality system including an ICE catheter, a general/TTE probe and a RF ablation catheter.

FIG. 41 is a schematic showing the noise detection/correction system 800 in FIG. 34 working in a multiple-modality system including an ICE catheter, a TEE catheter and a RF ablation catheter.

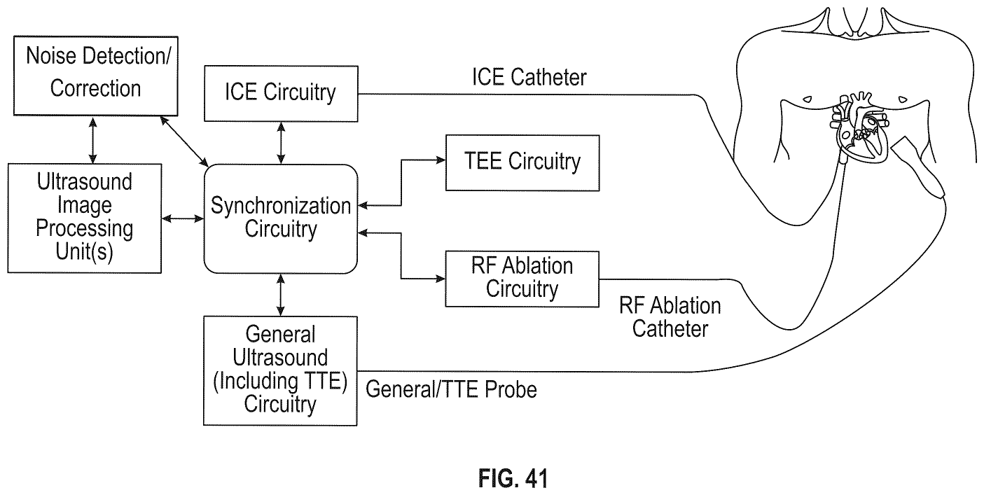

FIG. 42 is a schematic showing the noise detection/correction system 800 in FIG. 34 working in a multiple-modality system including a TEE catheter, a general/TTE probe and a RF ablation catheter.

### DETAILED DESCRIPTION OF CERTAIN INVENTIVE ASPECTS

Ever since the invention of the modern disposable catheter in 1940s, it has enjoyed rapid growth and wide expansion in the medical fields. To date, catheters have found applications in treating cardiovascular, urological, gastrointestinal, neurovascular, and ophthalmic diseases. Especially in the area of treating cardiovascular diseases there exist many catheter modalities. Among them are intracardiac echocardiography (ICE), intravascular ultrasound (IVUS), radiofrequency (RF) ablation, and fractional flow reserve (FFR). Intracardiac echocardiography (ICE) adopts a microscopic ultrasound array to realize direct imaging of anatomical cardiac structures in heart. It greatly helps procedures such as radiofrequency (RF) ablation. Intravascular ultrasound (IVUS), on the other hand, uses a microscopic ultrasonic array to generate images of a blood vessel, i.e., coronary artery. Radiofrequency (RF) ablation applies heat to destroy diseased tissue in order to stop pain. And fractional flow reserve (FFR) adopts a pressure sensor to measure pressure difference across a coronary artery stenosis to determine its effect on oxygen delivery to the heart tissue.

A catheter modality device normally comprises two parts, a hardware system having electronics with software build-in to send control signals to manipulate the procedure and to acquire and process data for display and operation, and a disposable catheter which usually includes a catheter tip, a handle to operate the catheter, and an electrical connector to connect to the hardware system. Generally, the hardware system is disposed on a wheeled platform so that the device can be easily moved to different places, for example, a lab, a procedure room, or a storage room. Different modalities are developed to treat different diseases. And even a single modality can have different devices developed by different companies potentially adopting different technologies for signal processing and data computing. The result is that each device has its own hardware system and catheter, and no two catheter devices share common hardware. Therefore, a hospital normally needs a large storage room to house and maintain all types of medical equipment, including many catheter devices, and a lab or procedure room needs to be large enough to hold at least few devices. In underdeveloped countries and areas, however, hospitals are usually tight of space. In certain hospitals, their lab or procedure room can even be too small to hold two catheter devices the same time. This may bring challenges to certain procedures. For example, the RF ablation procedure is usually accompanied by intracardiac echocardiography (ICE) to acquire real time imaging of the internal heart surface for accurately positioning the RF ablation catheter.

Therefore, there is need to develop a hardware system that can control the operation and process data for multiple catheter modalities. Preferably, the multiple-modality hardware system is small in size for better portability. Another potential feature of the multiple-modality hardware system is that it connects wirelessly to control console, remote control, display or displays and other peripheral equipment. In this way, the multiple-modality hardware system can be moved around without moving the peripheral equipment and can be installed or stored without occupying much space.

### List of Certain Components

The following is a list of certain components that are described and enumerated in this disclosure in reference to the above-listed figures. However, any aspect of the devices illustrated in the figures, whether or not named out separately herein, can form a portion of various embodiments of the invention and may provide basis for claim limitation relating to such aspects, with or without additional description. Generally, herein, reference to a “proximal” portion indicates a portion/component that is positioned closest to the patient when the device/component is in use (e.g., during an intracardiac echocardiography procedure), and reference to a “distal” portion indicates a portion/component that is positioned farther from the patient when the device/component is in use. The enumerated components include:

### List of Certain Parts

100 embodiment of wireless multiple-modality system
101 battery pack
103 wireless module
105 computing module
107 ICE and IVUS ultrasound circuitry module
121 wireless signal
123 control console
124 remote control(s)
125 display(s)
131 link cable
135 universal catheter handle
137 outer cylindrical shell
139 inner cylindrical body
141 control knobs
143 support feet
145 pushrods
147 control buttons
148 internal space
149 contact pads
151 catheter
155 catheter tip
137 cylindrical handle shell
139 rotatable handle body
141 control knobs
143 base support
145 pushrods
147 buttons
149 contact pads
200 embodiment of wireless multiple-modality system in handle
201 battery pack
203 wireless module
205 computing module
207 ICE and IVUS ultrasound circuitry module
221 wireless signal
300 embodiment of wireless multiple-modality system
301 battery pack
303 wireless module
305 computing module
307 ICE and IVUS ultrasound circuitry module
307A ICE ultrasound circuitry
308 RF ablation circuitry module
321 wireless signal
331 link cable
351 catheter
351A ICE catheter
353A ICE catheter tip
351C RF ablation catheter
355 catheter tip
361 synchronization circuitry
365 scheduler
371 synchronization circuitry
373 ultrasound image processing unit
381 synchronization circuitry
383 ultrasound image processing unit
385 scheduler
400 embodiment of wireless multiple-modality system in handle
401 battery pack
403 wireless module
405 computing module
407 ICE and IVUS ultrasound circuitry module
408 RF ablation circuitry module
421 wireless signal
451 catheter
455 catheter tip
500 embodiment of wireless multiple-modality system
501 battery pack
503 wireless module
505 computing module
507 ICE and IVUS ultrasound circuitry module
508 RF ablation circuitry module
509 FFR interface circuitry module
521 wireless signal
531 link cable
551 catheter
555 catheter tip
600 embodiment of wireless multiple-modality system in handle
601 battery pack
603 wireless module
605 computing module
607 ICE and IVUS ultrasound circuitry module
608 RF ablation circuitry module
609 FFR interface module
621 wireless signal
651 catheter
655 catheter tip
700 lesion inference layer
702 ICE Imaging
704 ultrasound image frame
706 display images live to user
708 lesion detector CNN
710 lesion detection decision
712 decision to improve ICE focus and power
714 ultrasound imaging controls
716 ablation power and pulse optimizer
718 decision to adjust RF ablation controls
720 RF ablation controls
722 lesion tracker RNN
724 decision to acquire DL guided lesion image
726 servo control to tip rotation
728 decision if full lesion is acquired
730 ICE tip rotation control
800 noise detection/correction system
802 ICE, TTE and TEE imaging controls
804 ICE
806 TEE
808 TTE
810 RF ablation
812 ICE image frame
814 noise detector
816 abnormal ICE noise decision
818 intra system detection noise response
820 undetermined source detected noise response
822 noise frequency analysis and auto filter generation for de-noising
824 decision to adjust active ultrasound control parameters
826 decision to adjust RF ablation controls
826 RF ablation controls

### Wireless Multiple-Modality System

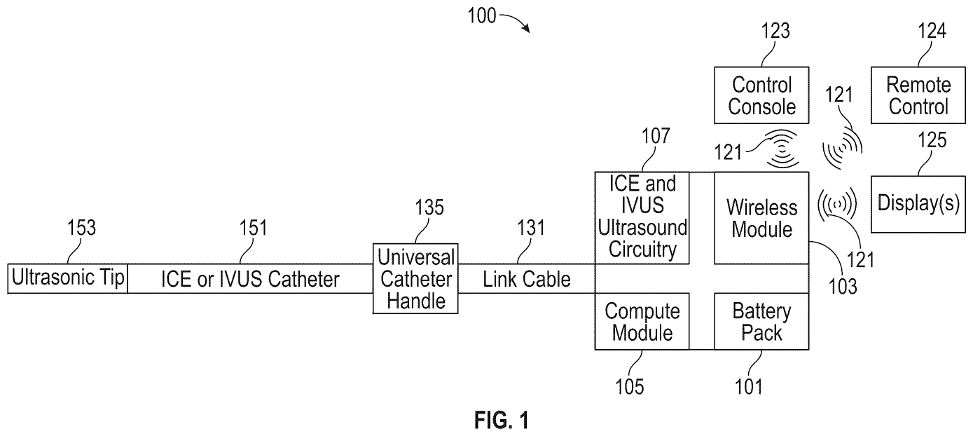

Referring now to the figures, FIG. 1 is a schematic illustrating an example embodiment of a multiple-modality system 100 connected to a catheter 151 having a catheter tip 153 through a link cable 131 and a universal catheter handle 135. The multiple-modality system 100 comprises modules made of PCBs and electronic components, including a battery pack 101, a wireless module 103, a computing module 105, and an ICE/IVUS circuitry module 107. These modules and components are assembled in a small housing or chassis for easy storage and transportation. The battery pack 101, preferably disposed in a compartment in the housing or chassis, supplies power to run the hardware system, the catheter 151 and the universal catheter handle 135. Preferably, the battery pack 101 consists of a rechargeable battery set and has a battery charger disposed in the chassis. In this way the multiple-modality system 100 can be powered by AC from a wall outlet or powered by the battery pack 101 when standalone. When fully charged and running standalone, it is preferred that the battery pack 101 can support the multiple-modality system 100 to operate for at least 4 hours, and more preferably more than 6 hours. The battery pack 101 can also adopt non-rechargeable batteries. In this case, the multiple-modality system 100 preferably has built-in AC power connection in case the non-rechargeable batteries do not contain enough power to support a procedure.

The wireless module 103 has electronics and software loaded thereon to connect the multiple-modality system 100 with a display or displays 125, a remote control or remote controls 124, a control console 123, which may be a laptop computer or a tablet computer, and potentially other peripheral accessories, through wireless signal 121. The wireless communication is preferably based on a short-range wireless protocol, such as Bluetooth®, WiFi, or ZigBee®.

The computing module 105 includes microelectronics, such as ASIC, CPU, GPU or FPGA, and other electrical components, and software loaded on a memory chip or a hard drive to process image data acquired from and to send control commands to the catheter 151 or handle 135. The modality circuitry module 107, on the other hand, funnels data between the catheter 151 and the computing module 105. When it receives data from the catheter 151, the modality circuitry module 107 may carry out some basic data processing, i.e., data conversion, before sending the data to the computing module 105. The computing module 105 then performs tasks such as graphics processing and data analysis, and sends the processed data to the control console 123, remote control(s) 124 and the display(s) 125. Upon reviewing the displayed graphics and the analyzed data results, the physician who operates the multiple-modality system 100 makes decision to send out commands from the control console 123 to perform certain actions at the catheter tip 153 or to perform certain further data analysis.

The modules of the multiple-modality system 100 shown in FIG. 1 are preferably assembled in a housing or chassis that is sized equal to or smaller than 15 inch×12 inch×6 inch, and preferably equal to or smaller than 12 inch×10 inch×4 inch. Such a multiple-modality system in a small chassis can be carried around easily. It can be stored on a shelf or in a cabinet in a storage room, taking only a small space. And it can easily be installed in a lab or procedure room even for those hospitals with tight space. If needed, it can be stacked with other equipment. Furthermore, the wireless connection capability allows the multiple-modality system 100 to share peripheral equipment, such as control console 123, remote control(s) 124 and display(s) 125, with other devices in the room. As such, moving the multiple-modality system 100 is usually not accompanied by moving peripheral equipment.

The modules of the multiple-modality system 100, including the battery pack 101, the wireless module 103, the computing module 105 and the modality circuitry module 107, can be built into a plurality of PCBs and electronic components assembled to the chassis, with connector(s) on a chassis wall to connect to the link cable 131. In other embodiments, the electronics of the computing module 105 and the wireless module 103 can be mounted on a main PCB that is installed in the chassis, and the modality circuitry module 107 is then connected to the main PCB as a child PCB. In this case, the modality circuitry module 107 child PCB may have a connector or two connectors built on it. When assembled, the connector(s) on the modality circuitry module 107 child PCB is exposed through a wall on the chassis to connect to the link cable 131.

For optimal system performance, a combination of active and passive cooling methods is employed for thermal management. This may involve multiple sensors installed in the chassis of the multiple-modality system 100 to monitor temperature in order to achieve optimal power delivery management and heat removal.

The modality circuitry module 107 shown in FIG. 1 supports intracardiac echocardiography (ICE) and intravascular ultrasound (IVUS). Since both ICE and IVUS acquire images inside the body by transmitting ultrasound signals from ultrasonic arrays, they can have shared circuitry and electrical components. This makes it natural to build a common hardware to support both ICE and IVUS. The modality circuitry module 107 is designed in such a way that it allows the control console 123 to configure which catheter to support, the ICE, the IVUS, or both simultaneously. When used simultaneously, the ultrasound waves transmitted from one modality may interfere with the other modality. The result is noise and degraded ICE and IVUS ultrasound images. Therefore, the multiple-modality system 100 adopts a synchronization circuitry to minimize interference between the catheters for better functionality and optimal ultrasound image quality. And the system further uses feedback systems and algorithms to improve the co-functionality of the ICE and IVUS catheters. Specifically, the synchronization circuitry and software may have clocking and switching features to allow the ICE and IVUS catheters to split time. In this way, at a specific time, only one of the ICE or IVUS catheter transmits ultrasound and receives echoed ultrasound signal for image generation. In this way when integrated both catheters can acquire ultrasound images simultaneously. Preferably, the on and off cycle frequency of either ICE or IVUS due to the synchronization is more than 30 Hz, and more preferably 60 Hz, so that high quality images are attained. The synchronization circuitry will be further discussed later along with other embodiments.

The multiple-modality system 100 modality circuitry module 107 may have one shared connector to connect with either an ICE catheter or an IVUS catheter. And at a specific time, the connection can be detected by electronics on the modality circuitry module 105 or on the computing module 105. Or it can be configured by a physician on the control console 123. Or, the multiple-modality system may have a connector to connect an ICE catheter and another connector to connect an IVUS catheter. Whether one shared connector or two connectors are used, the multiple-modality system 100 shall be able to be configured to support either the ICE catheter or the IVUS catheter, or both. When both ICE and IVUS catheters are connected and operated, the synchronization circuitry discussed previously will greatly enhance ultrasound image quality. The modality circuitry module 107 may be built as a child PCB adapted to be connected to the main PCB that includes the computing module 105 and the wireless module 123.

In FIG. 1 the catheter 151 is connected to the multiple-modality system 100 through the catheter handle 135 and the link cable 131. The catheter handle 135 is a universal handle that can be connected to and operate different types of catheters, for example, the intracardiac echocardiography (ICE) catheter and the intravascular ultrasound (IVUS) catheter. It is to be noted that to support an ICE catheter and an IVUS catheter, two handles 135 are needed, one connected to each. Another way is to combine the ICE and IVUS into one catheter and operated by one catheter handle.

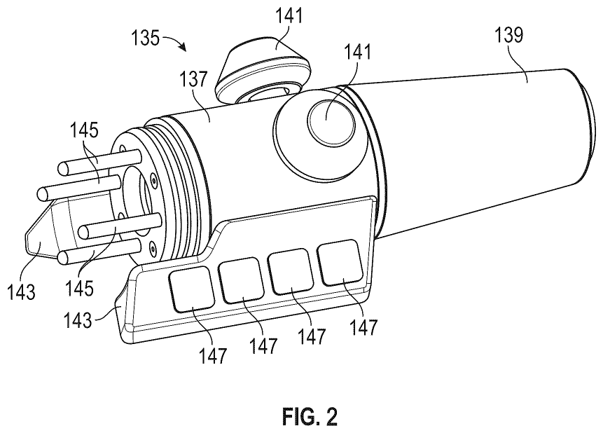

As shown in FIGS. 2 & 3, the universal catheter handle 135 comprises an outer cylindrical shell 137, an inner cylindrical body 139 residing inside and coaxial with the outer cylindrical shell 137, two support feet 143 that are connected to the outer cylindrical shell 137 and adapted to stand on a surface, and two control knobs 141 that can be rotated to operate the connected catheter. By rotating the control knobs 141, four pushrods 145 are actuated to tilt a swashplate (not shown) in contact with the pushrods, so as to bend and steer the catheter tip 153 shown in FIG. 1. And the inner cylindrical body 139 can be rotated to rotate the catheter 151 together with tip 153. Therefore, the tip bending and steering action from rotating the control knobs 141 and the tip rotating action from rotating the inner cylindrical body 139 ensures that the catheter tip 153 can enter any place in the body and point to any spot accurately. The push buttons 147 located on the support feet 143 may be used to control various functions of the catheter, including imaging functions.

The internal space 148 of the inner cylindrical body 139 is to hold the proximal end of the link cable 131 that is connected to the wireless multiple-modality system 100 at the distal end. In this disclosure, the proximal end means the end closer to the patient that the equipment serves, and the distal end means the end that is away from the patient. As the proximal end of the link cable 131 is inserted into the internal space 148, it has features to be locked in place while electrical connections are made to connect the link cable 139 to the contact pads 149 at the distal end of the inner cylindrical body 139. Meanwhile, the proximal end of the link cable 131 is electrically connected to an adaptor mechanism which is adapted to connect with the connector at the distal end of the catheter 151 to realize electrical connection between the link cable 131 and the catheter 151. In other embodiments, the proximal end of the link cable 131 has electrical connectors to directly connect to the distal end of the catheter 151. As such the adaptor mechanism in the handle 135 is not needed. The proximal end of the link cable 131 is keyed in the inner cylindrical body 139 by cross-sectional shape feature, so that rotating the inner cylindrical body 139 will bring the proximal end of the link cable 131 and the catheter 151 to rotate together.

The universal catheter handle 135 constructed as described above can be a reusable catheter handle. When the link cable 131 is unplugged from the handle 135 and the catheter 151 is removed, the catheter handle 135 can be sterilized either at room temperature or at elevated temperature. Then it can be used in a future procedure.

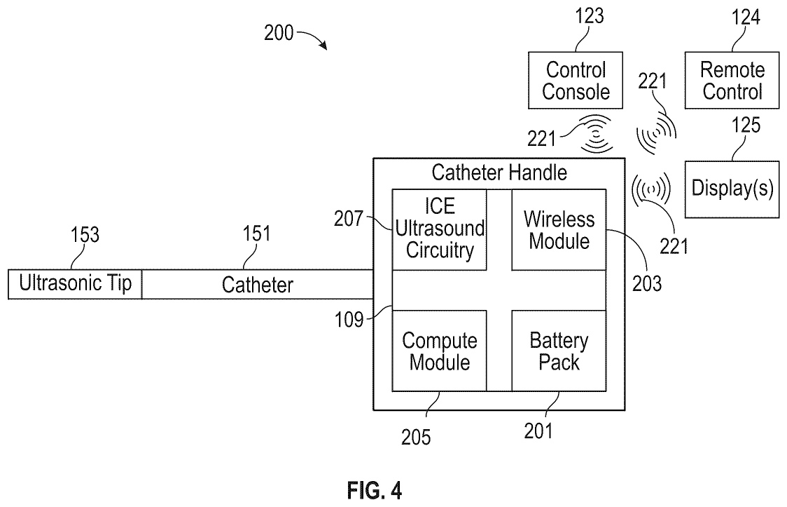

An alternative embodiment of the multiple-modality system 100 in FIG. 1 is illustrated in FIG. 4 as a multiple-modality system 200, which can include the similar modules of the multiple-modality system 100 shown in FIG. 1, except that the modules, including a battery pack 201, a wireless module 203, a computing module 205, and a modality circuitry module 207 are contained in a catheter handle. This means that the multiple-modality system 200 is essentially an integration of the modules in the multiple-modality system 100 and the universal catheter handle 135 in FIG. 1. As progress has been made in the semiconductor industry, component densities on IC chips and PCBs have been increasing tremendously in the past decades. Standing today, it is possible to build each module of the multiple-modality system on a high-density PCB, an IC chip, or even a chiplet. When all the components are assembled, the assembly can small enough to fit into the internal space provided by the catheter handle. For example, each of the computing module 205, wireless module 203 and modality circuitry module 207 can be designed and built as an IC chip. And the IC chips can be mounted on a high-density PCB to fit inside the handle. Or the functionalities of the modules can be distributed to a plurality of IC chips mounted on one or a few high-density PCBs small enough to fit inside the catheter handle. And the battery pack 201 is preferably a rechargeable cell battery potentially integrated with a charger, all fit inside the handle. The multiple-modality system 200 in a catheter handle communicates wirelessly with the control console 123, remote control(s) 124 and the display(s) 125, similar to the multiple-modality system 100 in FIG. 1. The multiple-modality system 200 is directly connected to the catheter 151 having a catheter tip 153. And the catheter 151 may be an ICE, or an IVUS, or both of them.

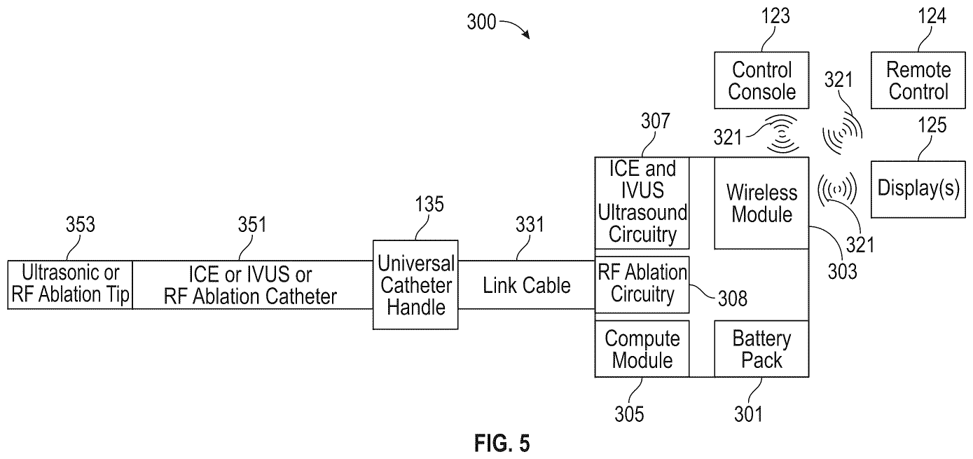

FIG. 5 shows a schematic of an embodiment of a multiple-modality system 300. In addition to the modality circuitry module 307 for the ICE and IVUS catheter modalities, the multiple-modality system 300 includes an RF ablation circuitry module 308 for a radiofrequency (RF) ablation modality. In other embodiments, the circuitry for RF ablation module 308 and the circuitry for the ICE and IVUS module 307 are integrated to form a combined circuitry to support all three modalities. Since ICE can generate ultrasonic image to accurately guide RF ablation, it makes great sense to integrate ICE and RF ablation modalities in one system. As with multiple-modality system 100 shown in FIG. 1, the multiple-modality system 300 further includes a battery pack 301, a wireless module 303, and a computing module 305. The multiple-modality system 300 may be contained in a chassis having one connector to connect to a link cable 331 which is in turn connected through a universal catheter handle 135 to a catheter 351 with a catheter tip 353 at the proximal end. In this case, the catheter 351 can be one of the ICE, IVUS and RF ablation catheters. Or, it can have up to 3 connectors each configured to connect to one of the ICE, IVUS and RF ablation catheters. As configured by the operating physician on the control console 123, the 3 catheter modalities can be operated individually or simultaneously. When two or three modalities are used simultaneously, a synchronization circuitry in the multiple-modality system 300, preferably included in the computing module 305, ensures that interference between the different modalities are minimized.

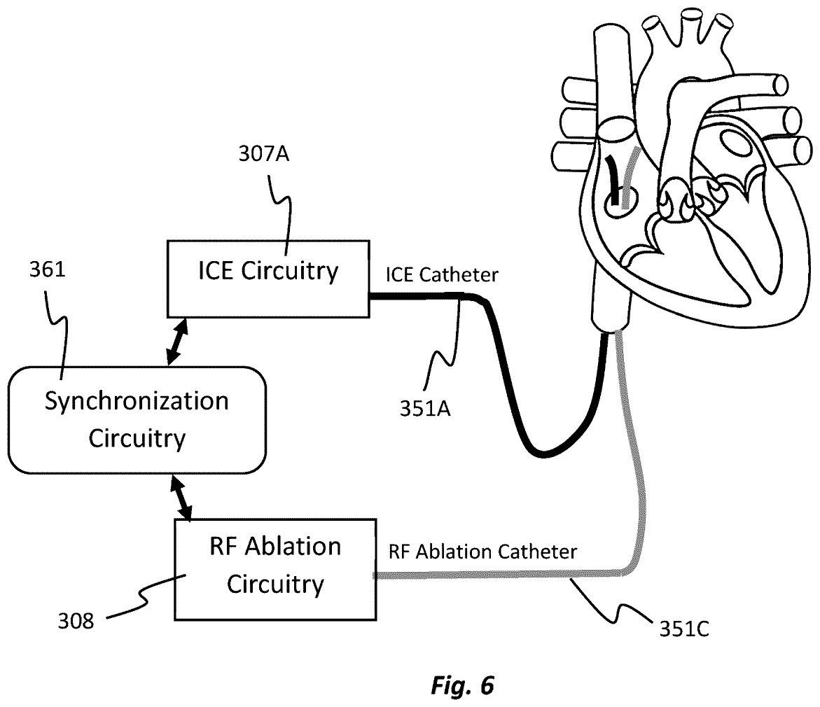

Since RF ablation is usually paired with intracardiac echocardiography (ICE), the synchronized operation of the two catheters is further described. During RF ablation, radiofrequency interference from the ablation catheter and ablation electronics to the ICE catheter and associated ultrasound system usually causes the quality of the ICE ultrasound images to significantly degrade. Usually, fluoroscopy and/or a sophisticated cardiac mapping system is needed to compensate for loss of image quality generated from the ICE catheter. The reverse can be true, the electrical interference may cause issues for RF ablation. However, when the two catheters share one hardware system, such as the multiple-modality system 300 shown in FIG. 5, this problem can be solved by having the two modalities controlled together via a synchronization control circuitry, as shown in FIG. 6.

Traditional RF ablation may employ RF frequencies in the range of about 400-500 kHz, whereas pulsed frequency ablation (PFA) may use pulse durations of a few 10ths of a millisecond, with a dead time of about 100 s of milliseconds between adjacent pulses. In both RF ablation and PRA, it is possible to arrange a few milliseconds of time for the ICE catheter to construct a high-quality ultrasound image. As shown in FIG. 6, a schematic of synchronization control of the ICE catheter 35A and RF ablation catheter 351C, a synchronization circuitry 361 is established to achieve the goal. The synchronization circuitry 361 typically consists of gating control that switches rapidly between the ICE circuitry 307A and the RF ablation circuitry 308, so that ICE ultrasound imaging frames can be acquired between the durations of either the sustained RF energy application (for traditional RF ablation catheter) or pulses of RF energy application (for emerging PF ablation catheters).

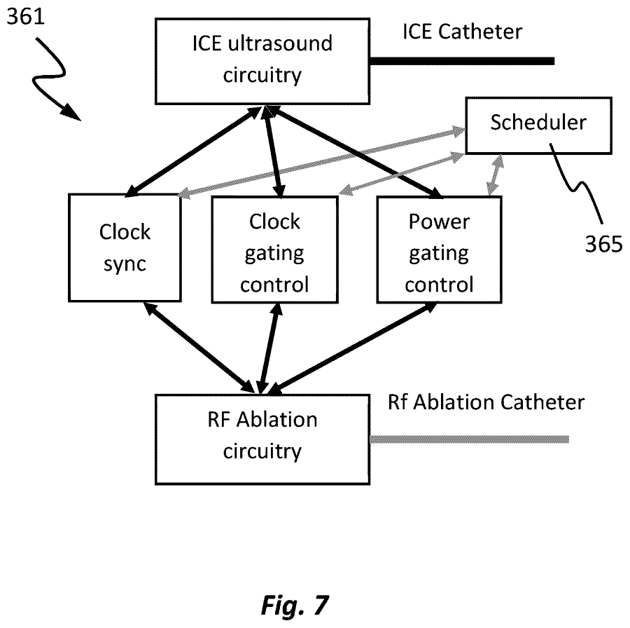

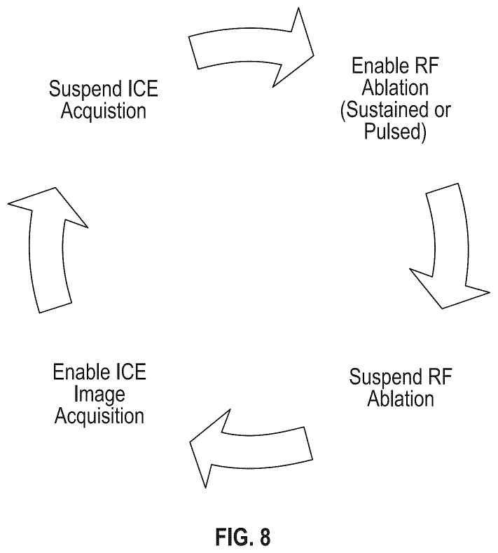

As shown in FIG. 7, the synchronization circuitry 361 includes a scheduler 365 to decide control transfer between the ICE and the RF ablation systems to form a repetitive cycle of operations between the ICE modality for ultrasonic image acquisition and the RF ablation modality for the ablation procedure, as shown in FIG. 8. Preferably, the frequency of the cycling is more than 30 Hz, and more preferably 60 Hz, so that high quality images are attained. The synchronization circuitry 361 further comprises a clock synchronizer to generate time signal for the catheter systems, a clock-gating controller to start, pause or stop the clocking and hence the dependent logical switching within each system, and a power-gating controller to start and stop power of each system.

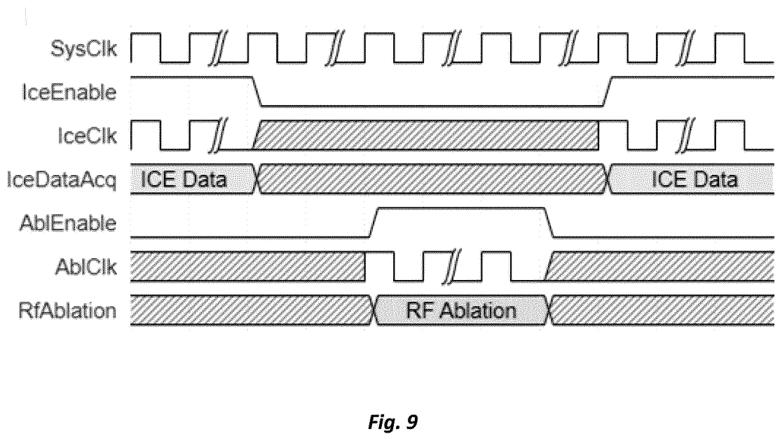

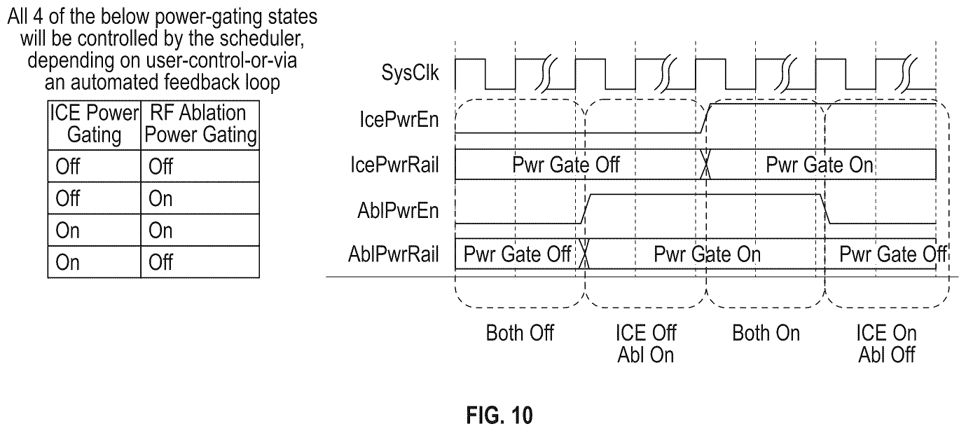

The clock-gating and the power-gating functions of the synchronization circuitry 361 in FIG. 6 are further illustrated through timing diagrams FIGS. 9 & 20. In both FIGS. 9 & 10, clock pulses are generated by the clock synchronizer in FIG. 7. In FIG. 9, the clock-gating controller in FIG. 7 switches on and off the ICE data acquisition and the RF ablation sequentially following sampled clock pulses and according to the scheduler's commands. And in FIG. 10, the power-gating controller in FIG. 7 turns on and off the power for the ICE and the RF ablation modalities, also according to the scheduler's commands. In FIG. 10 there are power-on overlaps between the ICE and RF ablation modality systems. But either the ICE or the RF ablation is turned on only when both its clock-gating and its power-gating are enabled. It is also to be noticed that the frequency of the clock pulses is significantly higher than the frequency of the cycles of the ICE and IVUS operations.

Referring back to FIG. 5, the multiple-modality system 300 communicates wirelessly with the control console 123, remote control(s) 124 and display(s) 125. And the multiple-modality system 300 can be made small in size, i.e., within 15 inch×12 inch×6 inch, and preferably within 12 inch×10 inch×4 inch, as with the multiple-modality system 100 shown in FIG. 1. And a combination of active and passive cooling methods involving temperature measurement and power delivery management is employed for thermal management.

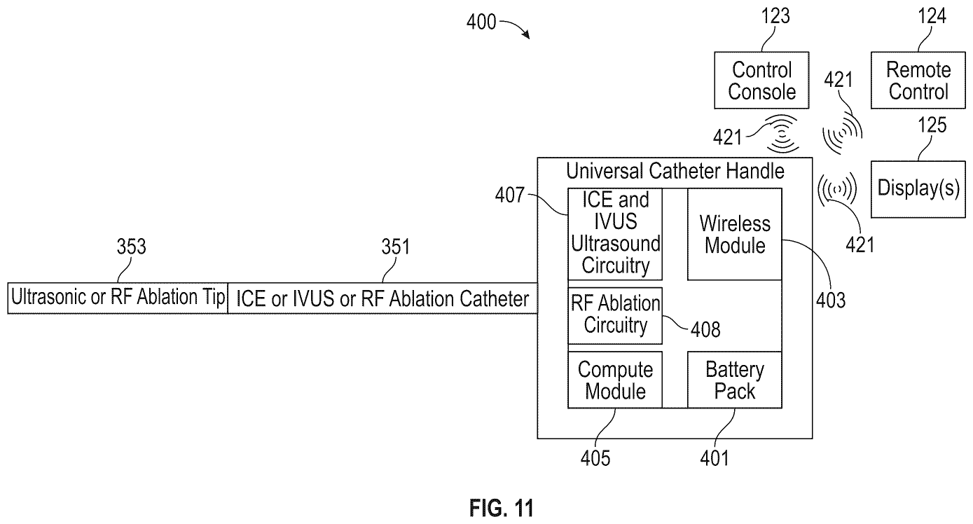

The same with the multiple-modality system 200 shown in FIG. 4, an alternative embodiment of the multiple-modality system 300 in FIG. 5 is illustrated in FIG. 11 as multiple-modality system 400. Components in the multiple-modality system 400 are built as ICs and high-density PCBs to fit inside the catheter handle. And it communicates wirelessly with the control console 123, remote control(s) 124 and the display(s) 125, similar to the multiple-modality system 300 in FIG. 5. The multiple-modality system 400 is directly connected to the catheter 351 having a catheter tip 353. The catheter 351 may be ICE, IVUS, or RF ablation, or any two of them, or all three of them.

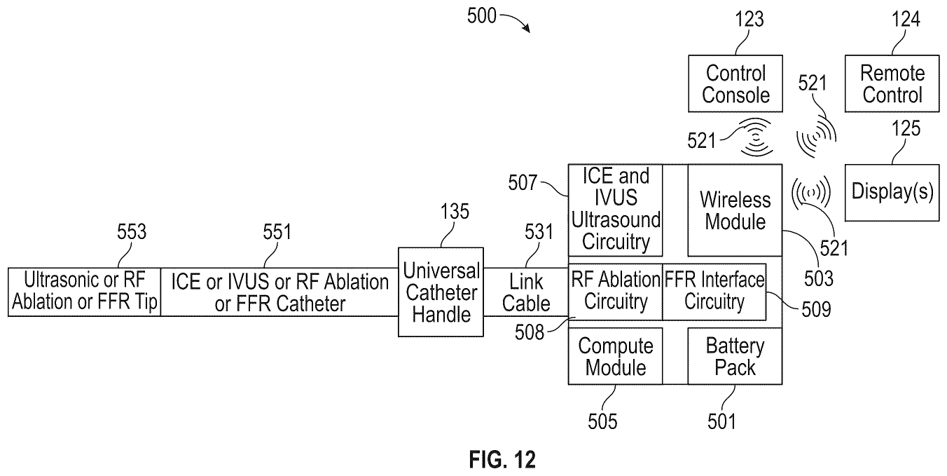

In FIG. 12, a multiple-modality system 500 includes 3 modality modules, an ICE and IVUS circuitry module 507, an RF ablation circuitry module 408, and an FFR interface circuitry module 509. In other embodiments, the circuitries for the three modules are combined as an integrated circuitry to support all four catheter modalities, including ICE, IVUS, RF ablation and FFR. In as much as the ICE and RF ablation modalities are paired to treat heart arrhythmia, IVUS and fractional flow reserve (FFR) can be complimentary in diagnosing whether the lesion is causing ischemia. Also, a synchronization circuitry described with FIGS. 6-10 will greatly help the performance of both.

As with multiple-modality system 100 shown in FIG. 1 and the multiple-modality system 300 in FIG. 5, the multiple-modality system 500 includes a battery pack 501, a wireless module 503, and a computing module 505. The multiple-modality system 500 may be contained in a chassis having one connector configurable to connect to a link cable 531 which is connected through a universal catheter handle 135 to a catheter 551 with a catheter tip 553 at the proximal end. Or, it can have up to 4 connectors each configured to connect to one of the ICE, IVUS, RF ablation, and FFR catheters. In this way, the 4 catheter modalities can be operated individually or simultaneously as configured and operated by the control console 123. As described with multiple-modality system 100 shown in FIG. 1 and the multiple-modality 300 shown in FIG. 5, the multiple-modality system 500 communicates wirelessly with the control console 123, remote control(s) 124 and display(s) 125. And it can be made small in size, i.e., within 15 inch×12 inch×6 inch, and preferably within 12 inch×10 inch×4 inch. A combination of active and passive cooling methods involving temperature measurement and power delivery management is employed for thermal management.

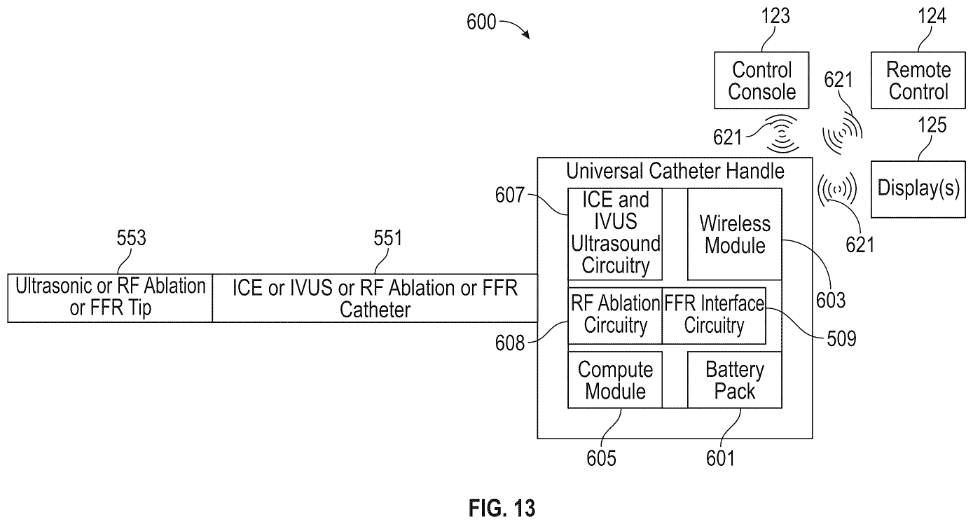

Again, the multiple-modality system 500 in FIG. 5 can be integrated with the universal catheter handle. This is illustrated in FIG. 13 as multiple-modality system 600. Components in the multiple-modality system 600 are built as ICs and high-density PCBs as compared with the modules and components in the multiple-modality system 500 to fit inside the catheter handle. And it communicates wirelessly with the control console 123, remote control(s) 124 and the display(s) 125, similar to the multiple-modality system 500 in FIG. 12. The multiple-modality system 600 is directly connected to the catheter 551 having a catheter tip 553. The catheter 551 may be an ICE, an IVUS, an RF ablation, or an FFR catheter, or combinations of two of them, three of them, or all four of them.

One of the ordinary in the art will appreciate that combinations of modalities in a multiple-modality system is not limited to catheter modality combinations illustrated in the referenced figures. Other catheter modalities and even probe modalities can be added, and different combinations can be established. In all cases, synchronization among the modalities is important to achieve optimal performances.

For example, other ultrasound imaging modalities can be added to the multiple-modality systems disclosed above. They may include transthoracic echocardiography (TTE) or general ultrasound probe which is used outside of the body to test the heart condition, and a transesophageal echocardiography (TEE) catheter which is inserted into the esophagus to obtain heart images. Therefore, including the catheters described previously, a multiple-modality system may include any two or more of the following modalities for controlling and processing data from an ICE catheter, an IVUS catheter, an RF ablation catheter, an FFR catheter, a TEE catheter, and/or TEE probe.

Explicitly for the modalities listed above, such a multiple modality system can support an ICE catheter in addition to any combination one or more of an IVUS catheter, an RF ablation catheter, an FFR interface catheter, a TEE catheter, and/or a general/TTE probe. And such a multiple modality system can support an IVUS catheter in addition to any combination of one or more of an RF ablation catheter, an FFR interface catheter, a TEE catheter, and/or a general/TTE probe. Further, such a multiple modality system can support an RF ablation catheter in addition to any combination of one or more of an of an FFR interface catheter, a TEE catheter, and a general/TTE probe. In addition to that, such a multiple modality system can support an FFR interference catheter in addition to a TEE catheter or a general/TTE probe. To exhaust the list, such a multiple-modality system can support a TEE catheter and a general/TTE probe.

### Synchronization of Multiple-Modality Systems for Optimal Performance

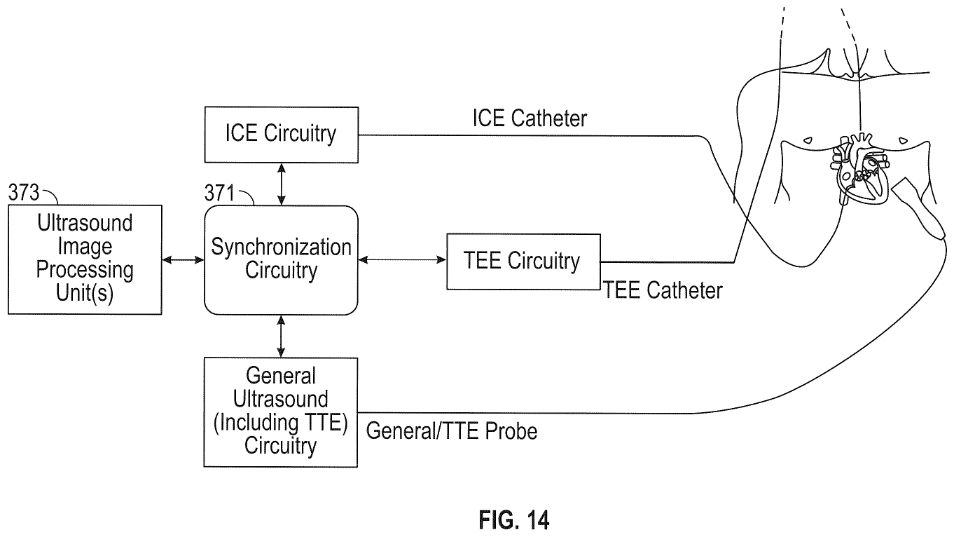

An example practice of synchronization among modalities not described above is reflected in FIG. 14, a schematic illustrating a synchronization circuitry 371 working with an ultrasound image processing system unit 373 to synchronize three ultrasound modalities, including an intracardiac echocardiography (ICE) catheter, a transthoracic echocardiography (TTE) probe which is used outside of the body to test the heart condition, and a transesophageal echocardiography (TEE) catheter which is inserted into the esophagus to obtain heart images. These three probe/catheters, either wired or wirelessly connected to a control console, remote control(s) and display(s), will ensure that ultrasound images of the heart are obtained from different directions. To date, no two ultrasound probe or catheter modalities can be used simultaneously on the same organ, since the ultrasonic interference between the systems results in significantly degraded images.

As discussed previously with FIGS. 6-10, this interference problem can be solved by having the two or more modality systems controlled together via a synchronization control circuitry that may either multiplex ultrasonic emissions in time across the probe/catheters or allow the ability to independently operate any one of them. FIG. 14 illustrates an intracardiac echocardiography (ICE) catheter in the heart, a transthoracic echocardiography (TTE) probe outside the body, and an external transesophageal echocardiography (TEE) catheter in the esophagus. All three are simultaneously used to image various features of the heart, potentially during a procedure, e.g., structural, electrophysiologic repair or device implant.

As shown in FIG. 14, such a synchronization circuitry 371 usually comprises a clock synchronizer to synchronize the clocks across two or more modalities, a clock-gating controller to start, pause or stop the clocking and hence the dependent logical switching within either system, and a power-gating controller to control momentary, longer, or complete power shutdown of either system. The synchronization circuitry 371 also includes a scheduler to decide control transfer among the three systems to establish a cycling among the three ultrasound echocardiography modalities, as shown in FIG. 14. As such high-quality ultrasound data is acquired and passed to the ultrasound image processing units 373. When two or more modalities are operating together, the synchronization circuitry 371 ensures that the modalities are turned on and off sequentially forming operation cycles and that at a specific time no more than one modality is turned on.

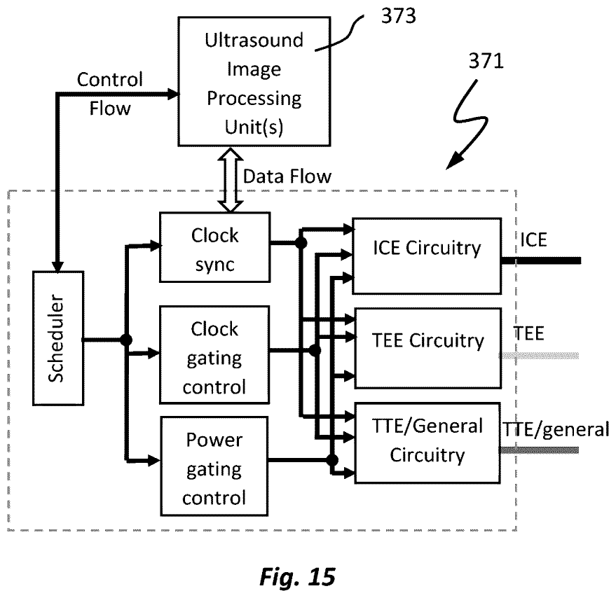

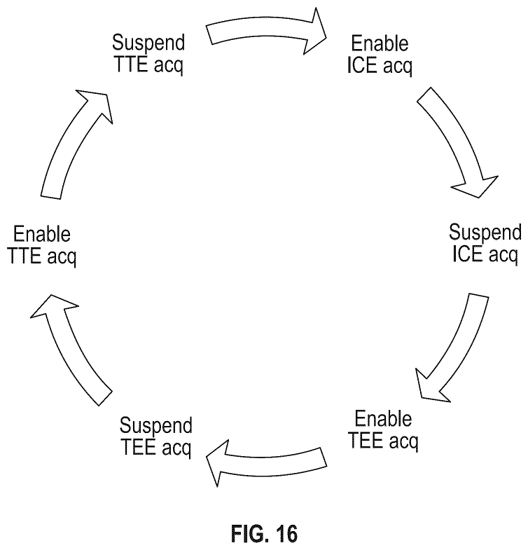

More specifically, as shown in FIG. 15, the synchronization circuitry 371 comprises a scheduler to control operation transfer among the modalities to form a repetitive cycle of operation for the ICE, the TEE, and the general/TTE modalities, a clock synchronizer to generate time signal for the catheter systems, a clock-gating controller to start, pause or stop the clocking and hence the dependent logical switching within each system, and a power-gating controller to start and stop power of each system. Preferably, the frequency of the operation cycle, which is shown in FIG. 16, is more than 30 Hz, and more preferably 60 Hz, so that high quality ultrasound images are attained.

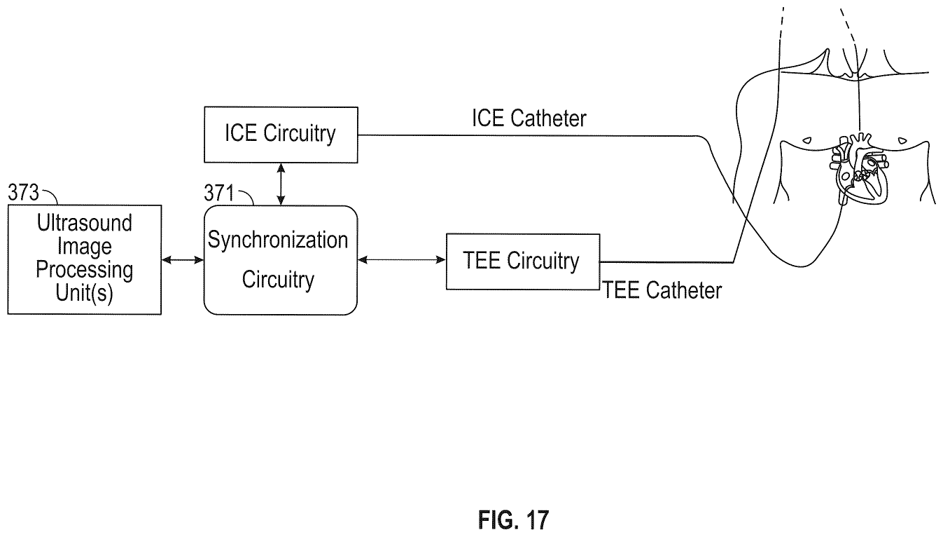

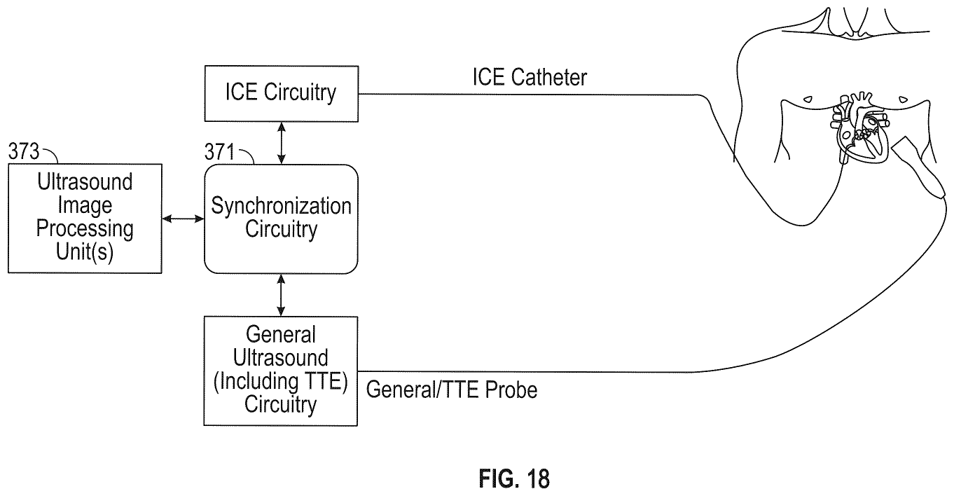

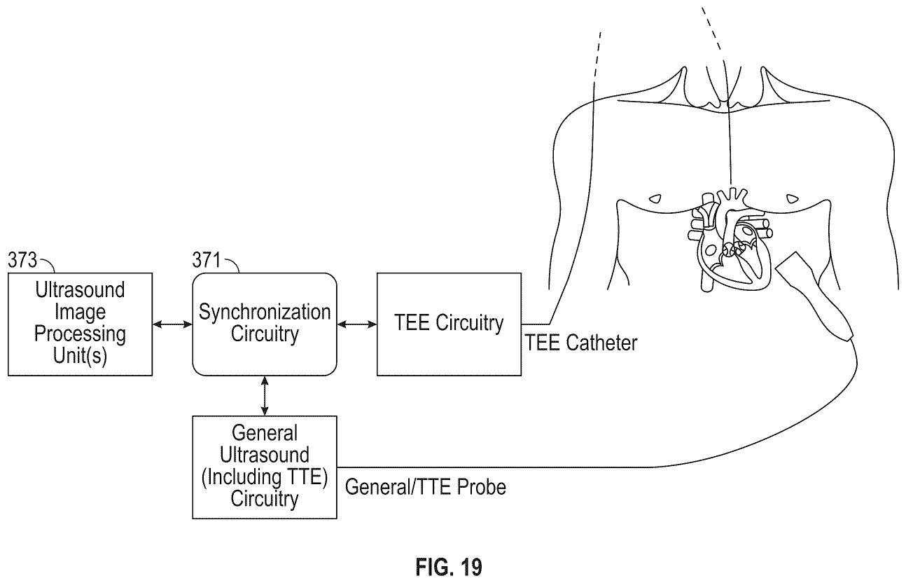

Potentially, two of the three ultrasound modalities in FIG. 14 can be operated simultaneously to get heart images. The combinations are shown in FIG. 17 for ICE and TEE modalities, in FIG. 18 for ICE and general/TTE modalities, and in FIG. 19 for TEE and general/TTE modalities. In all cases, the synchronization circuitry 371 ensures that the two modalities are turned on and off cyclically and that at a specific time no more than one modality is running.

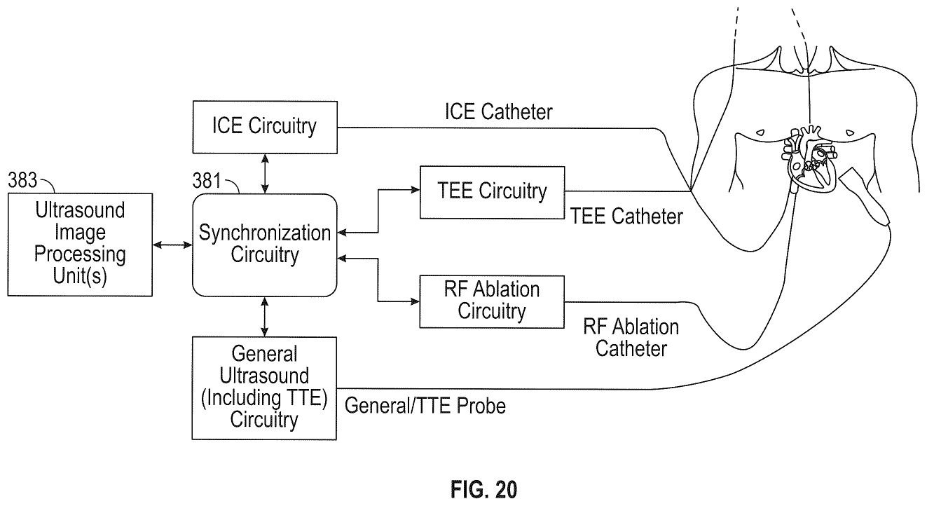

Another example practice of synchronization among modalities to reduce interference among ultrasonic and electronic systems is shown in FIG. 20, a schematic illustrating RF ablation catheter in the presence of more than one ultrasound modalities at the same time. In fact, three ultrasonic imaging systems, including an ICE catheter, a TTE/general probe, and a TEE catheter work in concert with RF ablation in a procedure. These modality systems can be either wired or wireless. In FIG. 20. A synchronization circuitry 381 works with an ultrasound image processing system 383 to synchronize three ultrasound modalities and the RF ablation catheter to get high quality ultrasound images.

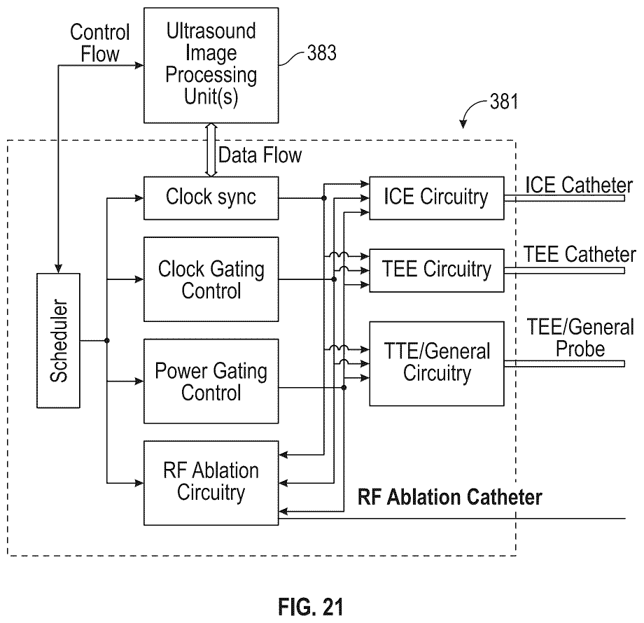

As shown in FIG. 21, it is possible to implement a circuitry to allow the 4 modalities to share time. As discussed previously with FIGS. 6-10 and FIG. 14-19, this problem of interference among modality systems can be solved by having the plurality of modalities controlled together via a synchronization control circuitry that will typically consist of gating control that switches rapidly in between the various ultrasound modality systems and the RF ablation modality system so that ultrasound imaging frames can be acquired between durations of either sustained RF energy application for traditional RF ablation modalities or pulses of RF energy application for emerging PF ablation catheter modality.

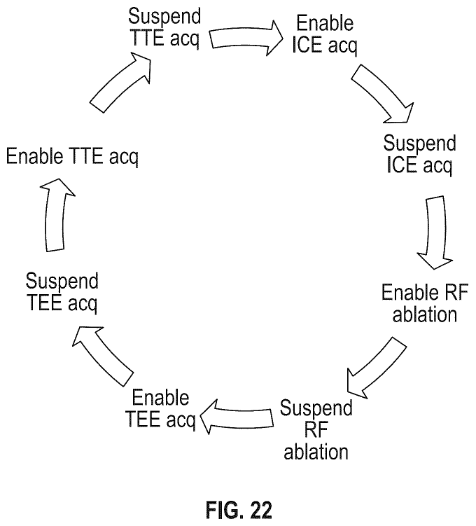

As shown in FIG. 21, such a synchronization circuitry 381 typically comprises a scheduler to decide operation transfer among the four systems and hence enable correct data flow into the ultrasound image processing units 383. The established cycling among the three ultrasound echocardiography modalities and the RF ablation catheter is shown in FIG. 22. The synchronization circuitry 381 also includes a clock synchronizer to synchronize to generate a clock pulse signal, a clock-gating controller to start, pause or stop the clocking and hence the dependent logical switching within each system, and a power-gating controller to control momentary, longer, or complete power shutdown of each system.

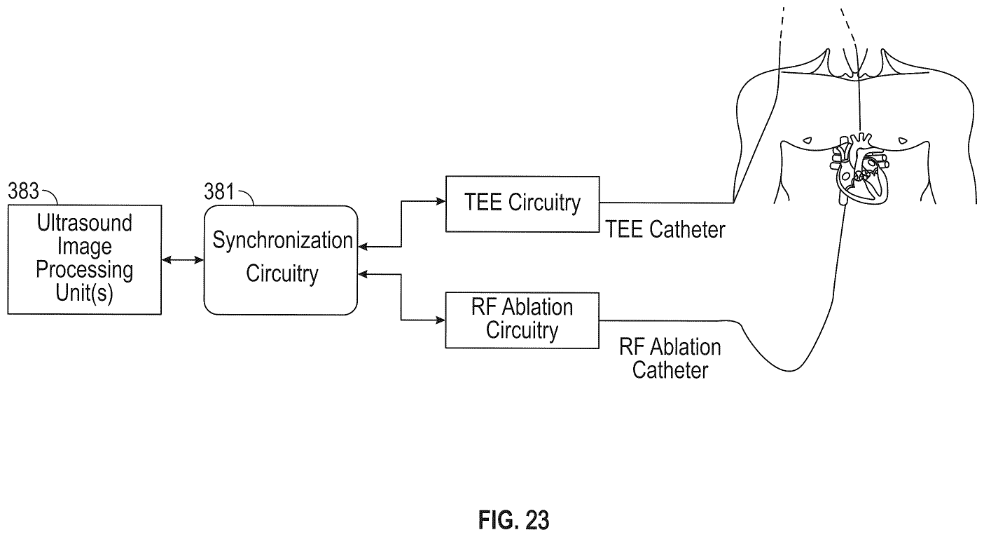

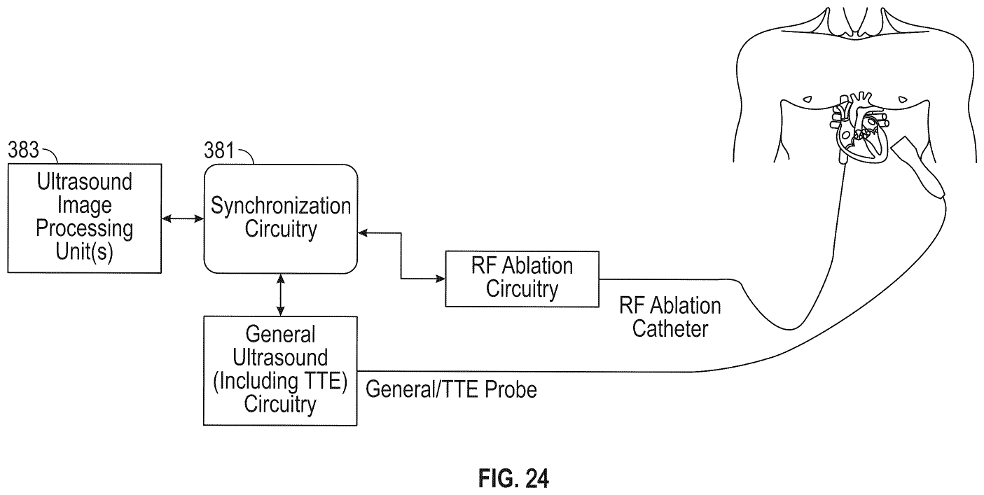

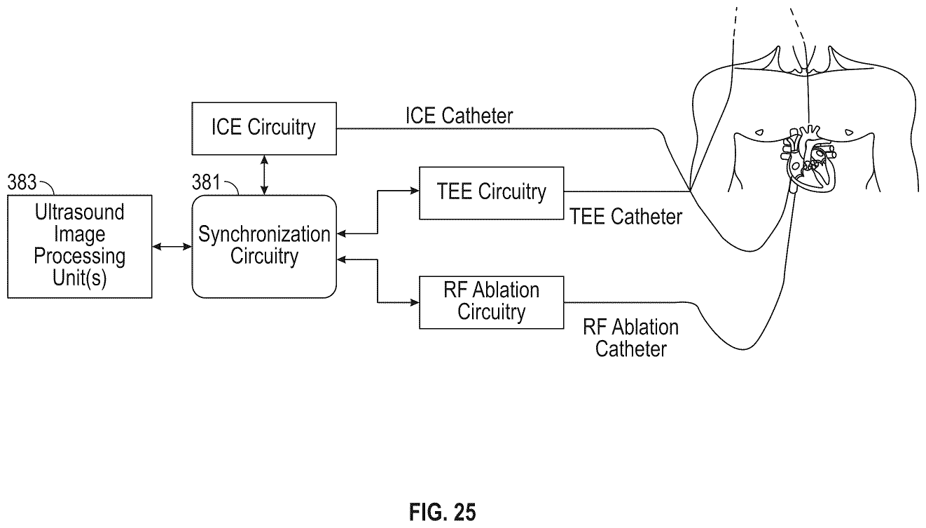

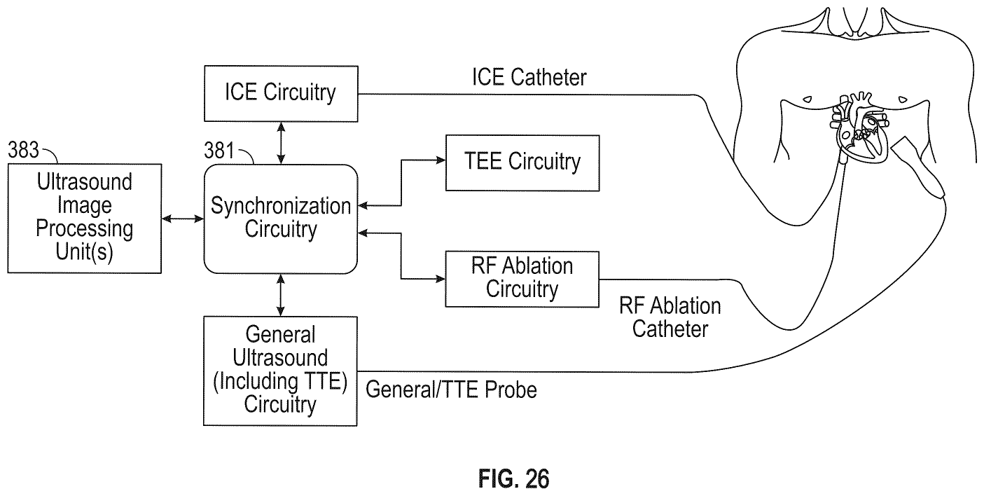

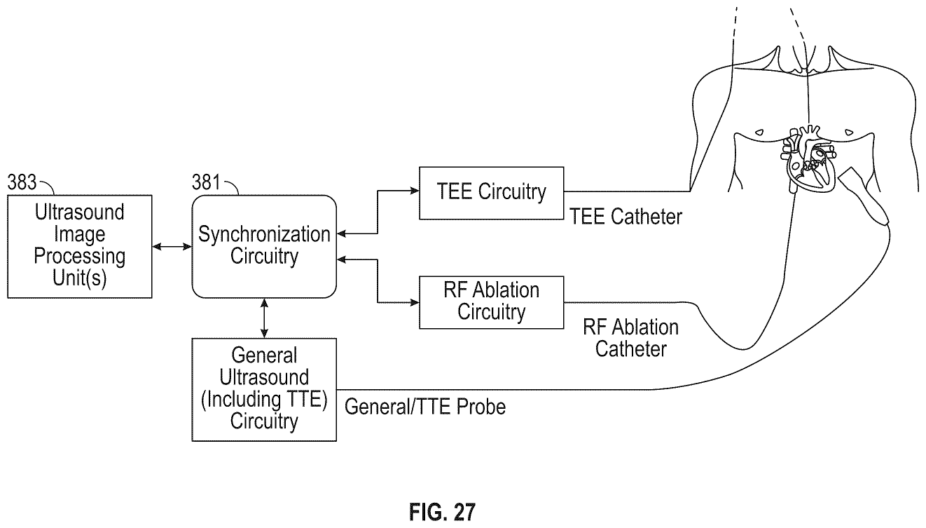

Except for synchronizing four modalities running the same time, the synchronization circuitry 381 can also synchronize two modalities, for example, the TEE catheter and the RF ablation catheter together shown in FIG. 23, and the RF ablation catheter and general/TTR probe together in FIG. 24, and synchronize three modalities, for example, the ICE catheter, the TEE catheter and the RF ablation catheter together in FIG. 25, the ICE catheter, the RF ablation catheter and the general/TTE probe together shown in FIG. 26, and the TEE catheter, the RF ablation catheter and the general/TTE probe shown in FIG. 27.

The synchronization circuitry can be implemented to any of the multiple-modality systems described previously. More broadly it can be implemented in any multiple-modality system supporting any two or more of the following modalities: the ICE catheter, the IVUS catheter, the RF ablation catheter, the FFR interface catheter, the TEE catheter, and/or the general/TEE probe.

Explicitly for the modalities listed above, the synchronization circuitry can be part of a multiple modality system supporting an ICE catheter in addition to any combination of an IVUS catheter, an RF ablation catheter, an FFR interface catheter, a TEE catheter, and/or a general/TTE probe. And the synchronization circuitry can be part of a multiple modality system supporting an IVUS catheter in addition to any combination of an RF ablation catheter, an FFR interface catheter, a TEE catheter, and/or a general/TTE probe. Further, the synchronization circuitry can be part of a multiple modality system supporting an FR ablation catheter in addition to any combination of an FFR interface catheter, a TEE catheter, and/or a general/TTE probe. In addition to that, the synchronization circuitry can be part of a multiple modality system supporting an FFR interference catheter in addition to a TEE catheter and/or a general/TTE probe. To exhaust the list, the synchronization circuitry can be part of a multiple modality system supporting a TEE catheter and/or a general/TTE probe.

### Deep Learning Inferencing-Based Ablation Control

Presently, each of the catheter and probe modalities on the market works standalone, with its own hardware, software and standard. If two or more modalities are involved in the same procedure, experts are relied on to read and analyze the data acquired from each of the modalities to make decision. And the individual who can master one modality may be not trained to operate another one. Human intelligence and interactions are key to use results from one modality to make adjustment for another. Therefore, it is nearly impossible to realize live feedback loop between two or more modalities based on the currently available technologies.

In this application, each of the multiple-modality systems disclosed can support two or more catheter or probe modalities with integrated electronics residing in the same housing. Such a multiple-modality system with synchronization circuitry implemented thereon opens the door to associate the different modalities involved in the same procedure, allowing the acquired data to be analyzed together and to form feedback loops on the multiple-modality system electronics or on the control console for optimization. Furthermore, deep-learning or artificial intelligence (AI) can be involved to drive the modality efficiency to a different level.

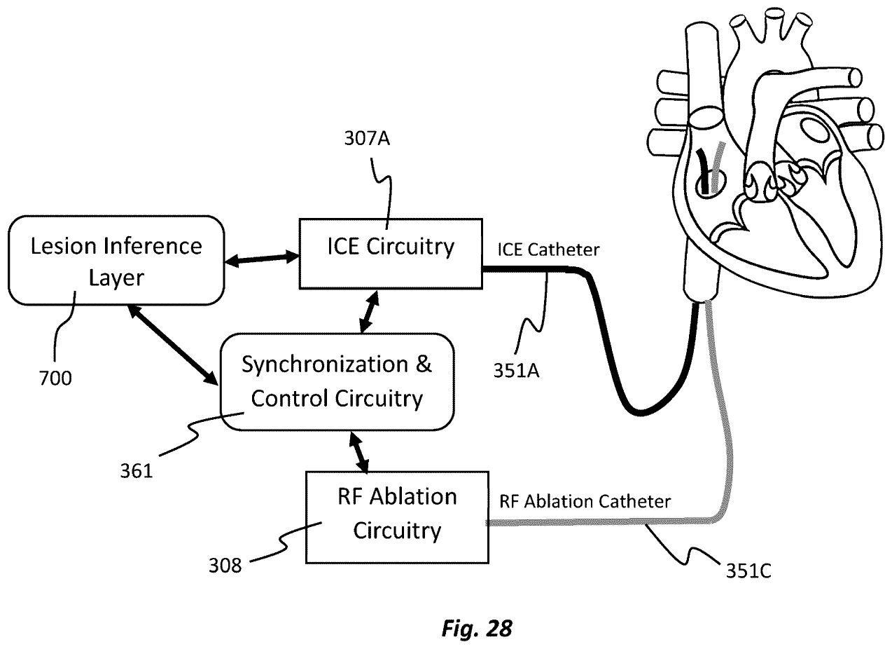

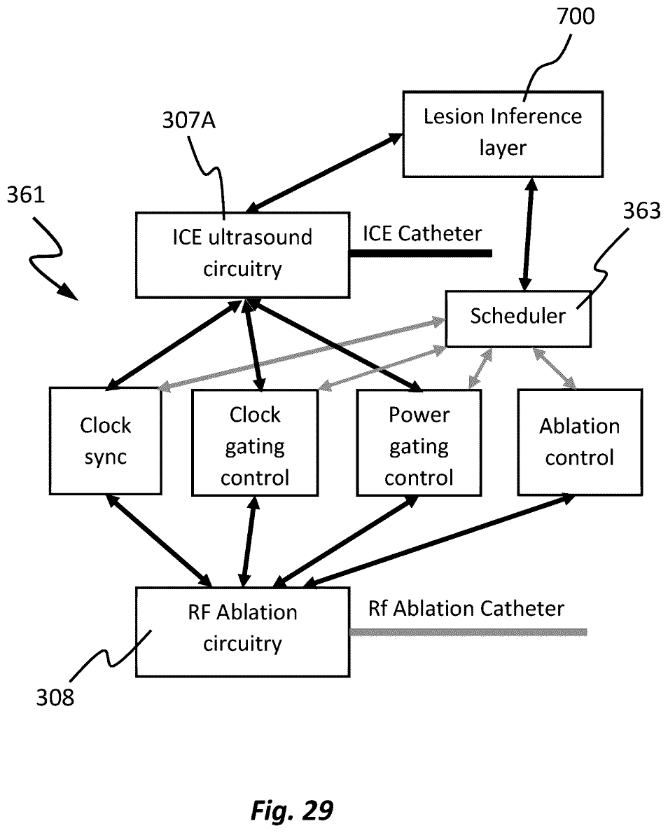

One example application is a lesion tracking deep-learning system for ICE and RF ablation modalities, as illustrated in FIG. 28. In the figure, a lesion inference layer 700 is built on top of the ICE circuitry 307A which supports an ICE catheter 351A and the synchronization circuitry 361 shown in FIG. 6 which is based on the multiple-modality system shown in FIG. 5 supporting both ICE and RF ablation modalities. In FIG. 29, the lesion inference layer 700 is shown to be connected to the ICE ultrasound circuitry 307A and the scheduler 365 which synchronizes the operations between the ICE ultrasound circuitry 307A and the RF ablation circuitry 308 in order to obtain high quality ultrasound images.

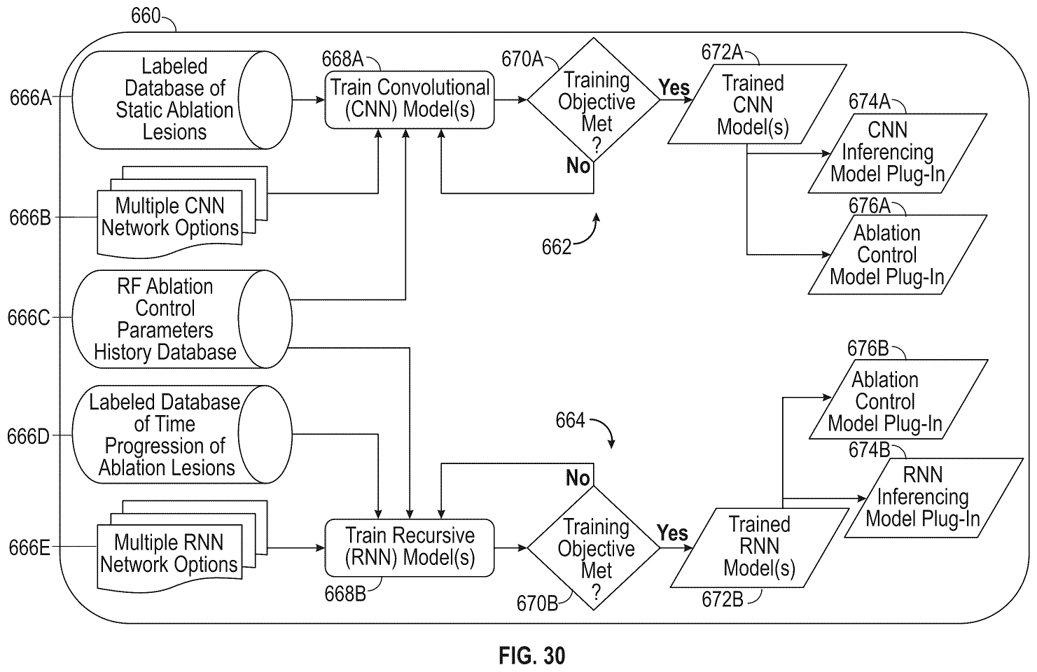

FIG. 30 is a flowchart to illustrate how deep-learning is realized for lesion inference. It involves both the convolution neural network and the recursive neural network to develop inference models.

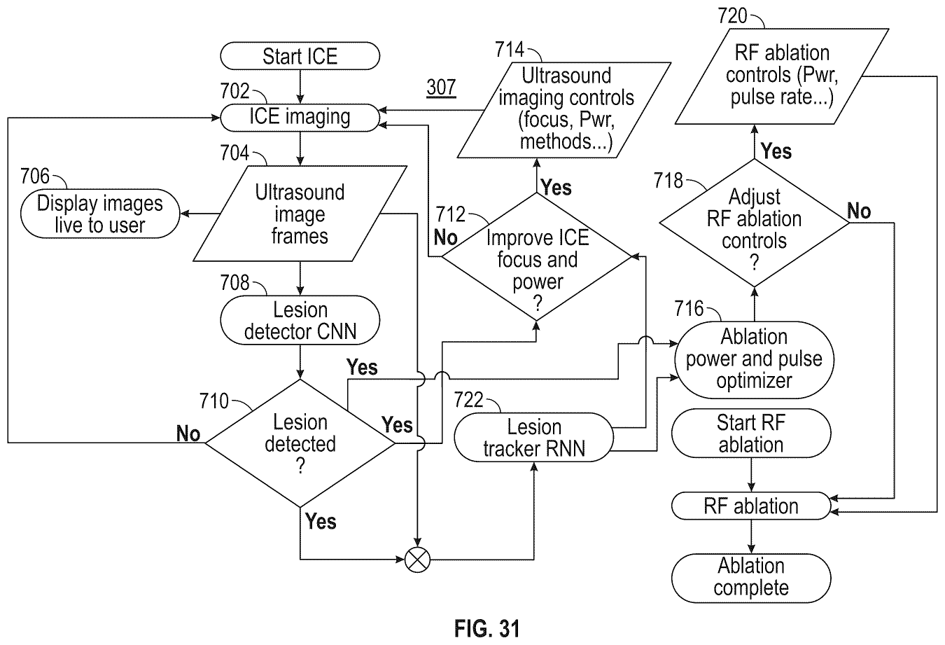

How the lesion inference layer 700 in FIGS. 29 & 30 works is illustrated by the flowchart shown in FIG. 31. This process can start by performing ICE imaging 702 and generating a number of ultrasound image frames 704 by an ICE catheter. The next step, the images can be communicated to a display in shown as live images to a user, 706, (e.g., medical practitioner performing the ICE catheter procedure). Some, or all, of the generated images can be provided to a lesion detection convolution neural network (CNN) 708 or other deep learning process where the images are analyzed for a lesion detection decision, 710, that is, the images are analyzed to determine if they contain a lesion. If not, the process can revert back to step 702, ICE imaging, where additional images can be generated by the ICE catheter.

If a lesion is detected, the process can determine if it should try to improve the quality of the images being generated, 712, for example, to improve the ICE focus of the images. In another example, a different level of power may be used to improve quality of images. If it determines not to improve the quality of the images, the process reverts back to step 702 where additional ICE images can be generated by the ICE catheter. If it determines to improve the imaging performed, adjustments may be made to ICE imaging controls 714 by dialing ultrasound image controls, i.e., the focus, power, a method of imaging, etc.

Also, if a lesion is detected, the process may lead to an ablation power and pulse optimizer 716, and adjustment the RF ablation controls, 718, can be decided. If the answer is yes, RF ablation controls 720 including power, pulse rate, and other parameters can be adjusted. In some embodiments, an indication is also provided relating to the distance between the RF ablation tip and the tissue being ablated which can help guide the practitioner in the positioning of the RF ablation catheter. The flowchart in FIG. 31 eventually leads to RF ablation to ablate away the lesion.

In addition, if a lesion is detected, the image may be sent to a lesion tracker recurrent neural network (RNN) 722. Information generated by the RNN can be used for adjusting the RF ablation power and pulse optimizer.

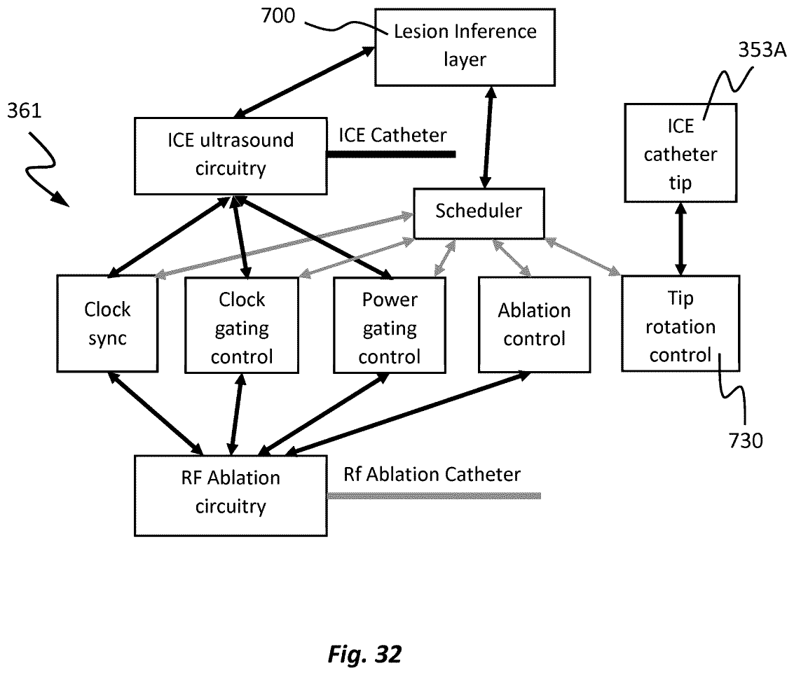

The lesion inference layer 700 shown in FIGS. 28-31 can be further developed to an automated lesion tracking deep-learning based system for ICE and RF ablation with synchronized operation for improved RF ablation outcome. FIG. 32 shows that the system in Figure includes an additional tip rotation control of ICE catheter tip 353A. This tip rotation control is preferably by automated servo control so that the whole process can proceed automatically to obtain high-quality volumetric lesion acquisition using 4D ICE array. Or it can be achieved through servo control of a rotatable tip about its central axis. Other embodiments may involve automated control of fluoro-arm rotation to achieve the same results.

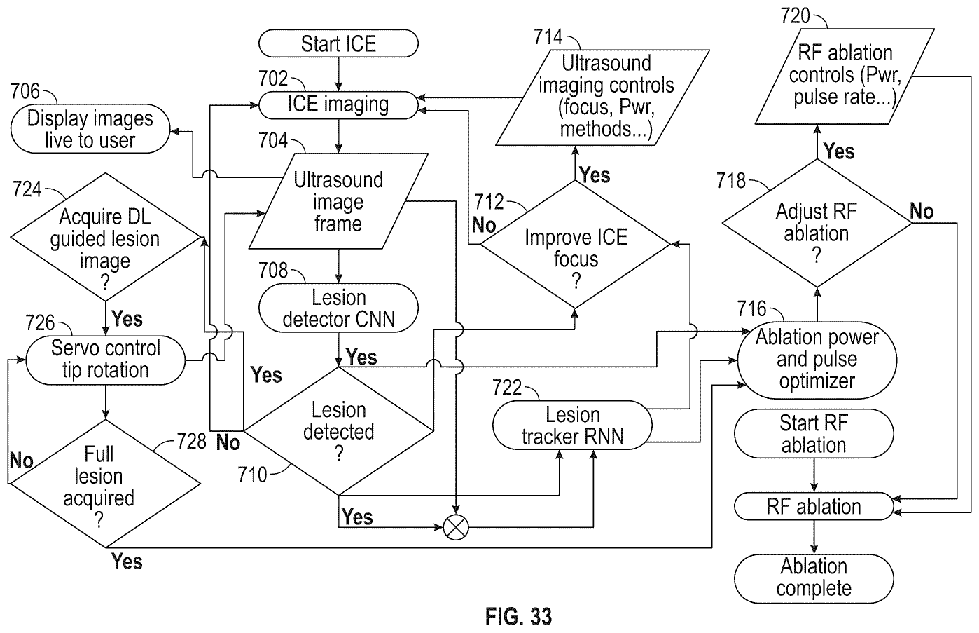

The flowchart of shown in FIG. 33 is for the automated lesion tracking deep-learning bases system for ICE imaging and RF ablation. In addition to the steps shown in FIG. 31, the flowchart further includes a portion involving onsite deep-learning (DL) after step 710, the lesion detection decision. If the answer from step 710 is yes, it leads to a decision to acquire DL guided lesion image. A positive answer further leads to a servo control tip rotation 726 to adjust the rotational positioning of the ICE catheter tip. Then the acquired image is evaluated to decide if full lesion is acquired, step 728. If the answer is yes, it proceeds to the ablation power and pulse optimizer 718 and eventually to RF ablation to eliminate the lesion.

The lesion inference layer 700 with synchronization circuitry described previously can be implemented to any of the multiple-modality systems disclosed in this application that includes an RF ablation catheter. More broadly it can be implemented with synchronization circuitry in any multiple-modality system supporting any one or more of the following modalities: the ICE catheter, the IVUS catheter, the FFR interface catheter, the TEE catheter, and/or the general/TEE probe.

Explicitly for the modalities listed above, the lesion inference layer together with the synchronization circuitry can be included in a multiple modality system having an RF ablation catheter to support an ICE catheter in addition to any combination of one or more of an IVUS catheter, an FFR interface catheter, a TEE catheter, and/or a general/TTE probe. And the lesion inference layer together with the synchronization circuitry can be included in a multiple modality system including an RF ablation catheter to support an IVUS catheter in addition to any combination of one or more of an FFR interface catheter, a TEE catheter, and/or a general/TTE probe. Further, the lesion inference layer together with the synchronization circuitry can be included in a multiple modality system including an RF ablation catheter to support an FFR interference catheter in addition to one or more of a TEE catheter and/or a general/TTE probe. In addition to that, the lesion inference layer together with the synchronization circuitry can be included in a multiple modality system including an RF ablation catheter can support one or more of a TEE catheter and/or a general/TTE probe.

### Noise Detection and Correction Scheme

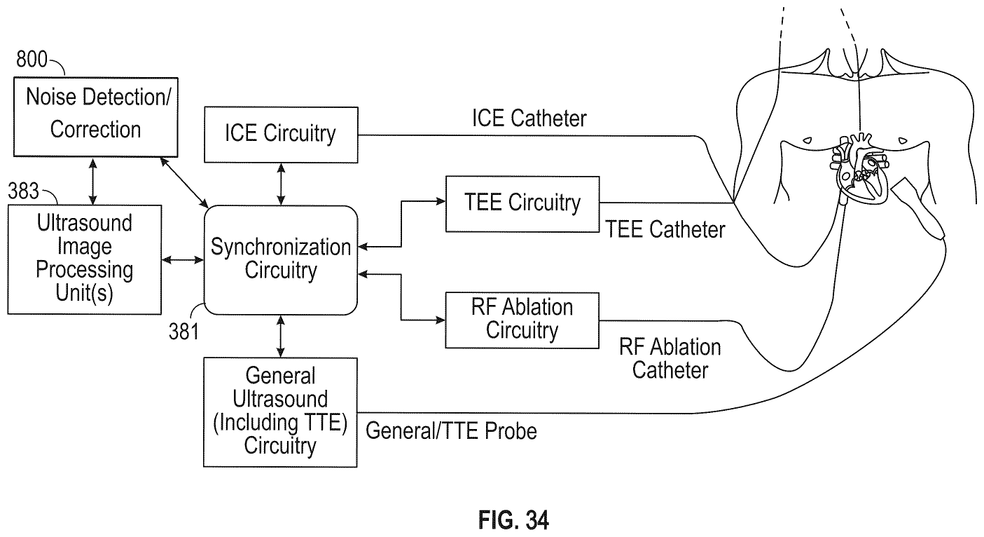

The synchronization system described previously with FIGS. 6-10 and FIGS. 14-27 can be further enhance by a noise reduction and correction system 800 shown in FIG. 34 in order to improve ultrasound image quality. In FIG. 34, the noise detection/correction system 800 is connected to the synchronization circuitry 381 and the ultrasound image processing unit(s) 383 shown in FIG. 20. FIG. 25 reveals more detailed connection between the noise detection/correction system 800 and other components, including a scheduler 385 which is part of the synchronization circuitry 381 to coordinate synchronization between the different catheter modalities. In turn, the scheduler is connected to the ultrasound image processing unit(s) 383. And the noise detection/correction system 800 is further connected to ultrasound modality circuitries, including the ICE catheter circuitry, the TEE catheter circuitry, and the TTE/general probe circuitry, to acquire noise signals.

To accomplish noise reduction the noise detection/correction system 800 keeps a running aggregate of the background and concurrent image quality characteristics, and upon detection of various types of noise, takes corrective actions. Depending on the source of the noise, different corrective actions may be taken. For example, a standard noise rejection filtering in software may be activated to filter out the general background noise. Or if the noise is determined to be likely from radiofrequency interference across various power regulators supplying the different systems, power circuitries of the different systems can be adjusted. If it is determined that the noise is caused by clocking interference across various systems, clock-gating of the different systems can be adjusted. And imaging frequency, focus and reconstruction methods can be adjusted to adapt filters specific to rejection of that noise, and then to revert back to prior state/method of image reconstruction. In addition, the noise detection/correction system 800 can look for noise detection on any one of the modality systems to then take pre-emptive noise reduction action across other co-functioning systems.

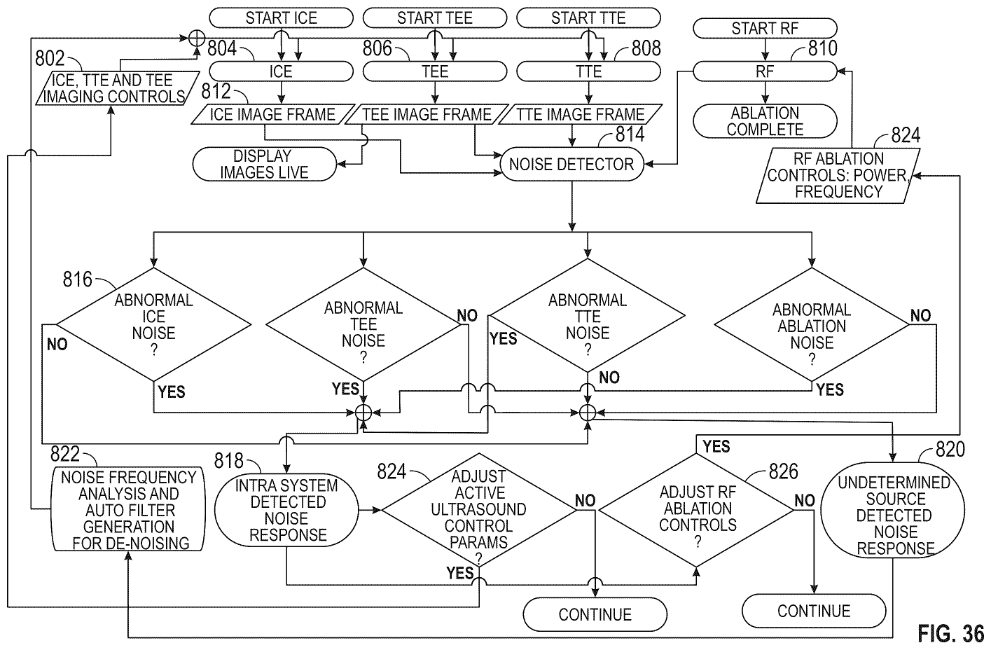

The process of how the noise detection/correction system 800 works is illustrated in FIG. 36, a flowchart showing the noise reduction schemes for ICE, TTE and TEE catheter/probe modalities for better RF ablation results. The left side of the chart shows an ICE, TTE and TEE image controls 802 for the best ultrasound image quality. And the right side is the RF ablation controls 824 for optimal ablation results. The flowchart includes steps to detect and correct noises for ICE 804, TEE 806, and TTE 808. Sine all three system are for ultrasound image acquiring, it is prudent to one of them as an example, i.e., the ICE, to illustrate all three. First, an ICE image frame 812 is taken and evaluated by a noise detector 814 for noise signal. Then an abnormal ICE noise decision 816 is determined according to the analysis of the noise detector 814. If the answer is yes, it leads the noise detection/correction system 800 to declare an intra system detected noise response 818, which further leads to a decision to adjust active ultrasound control parameters, step 824. A positive answer at this step will allow the system to revert back to the step 802 to adjust ICE imaging controls, and then to start the cycle of ICE image frame taken and evaluation for noise again. And a negative answer at step 824 allows the system to continue. Another action following the step 818, the intra system detected noise response announcement, is to decide to adjust RF ablation controls, step 826. If the answer is yes, the system will proceed to an adjustment of RF ablation controls, step 824. After that RF ablation can be carried out. At the step 816, if the answer is no to the abnormal ICE noise decision, the noise detection/correction system 800 declares an undetermined source detected noise response 820. After that, it leads to a noise frequency analysis and auto filter generation for denoising, step 822. And the system reverts back to the beginning to take and evaluate ICE image frame again.

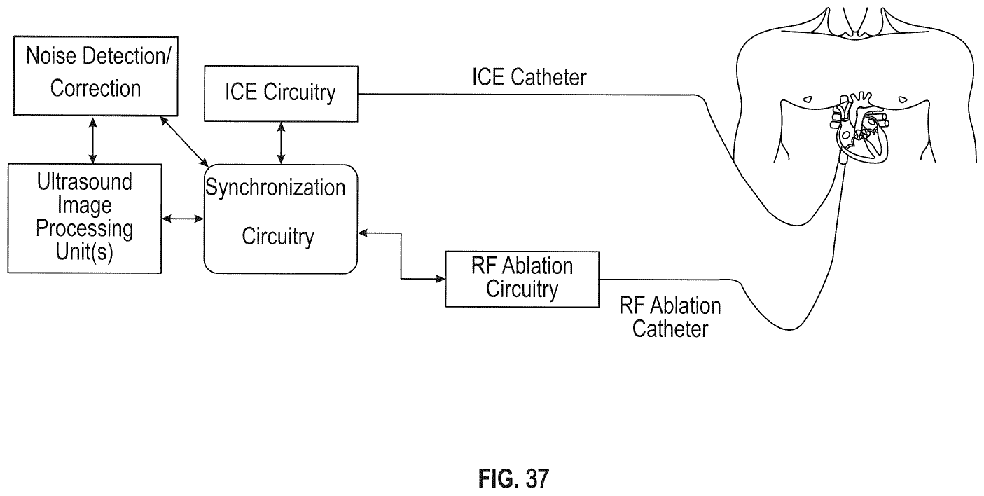

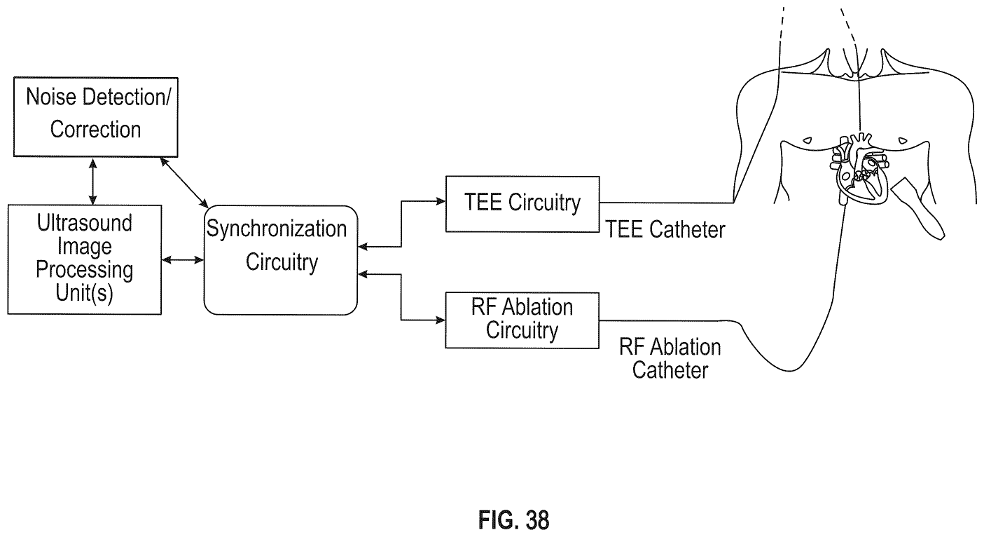

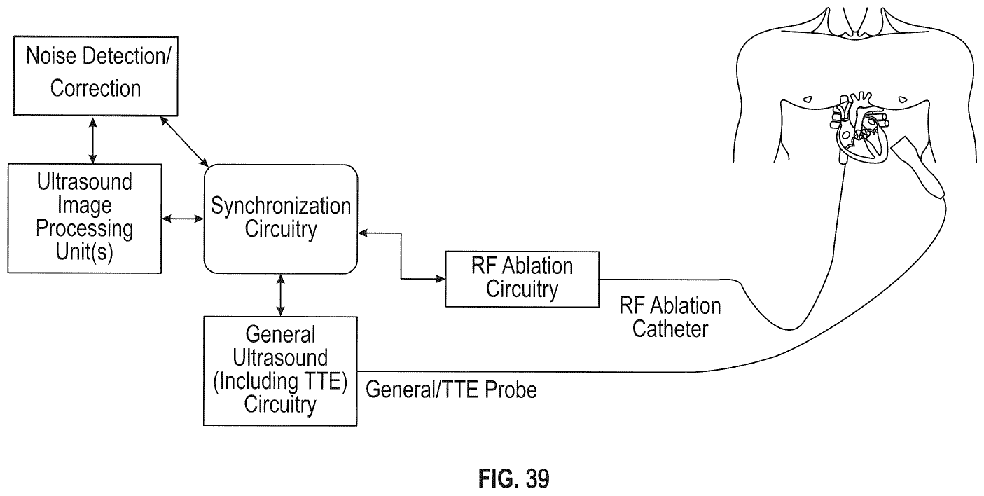

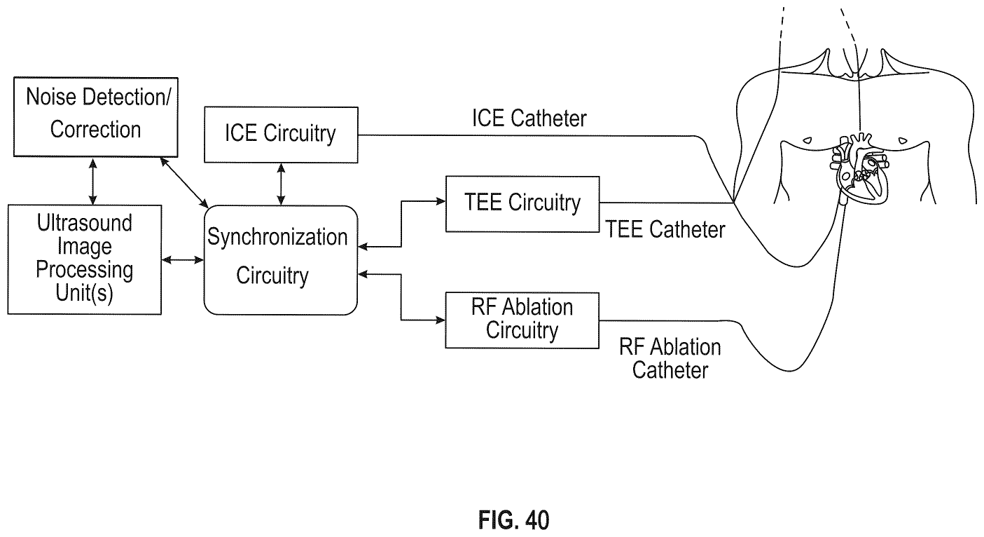

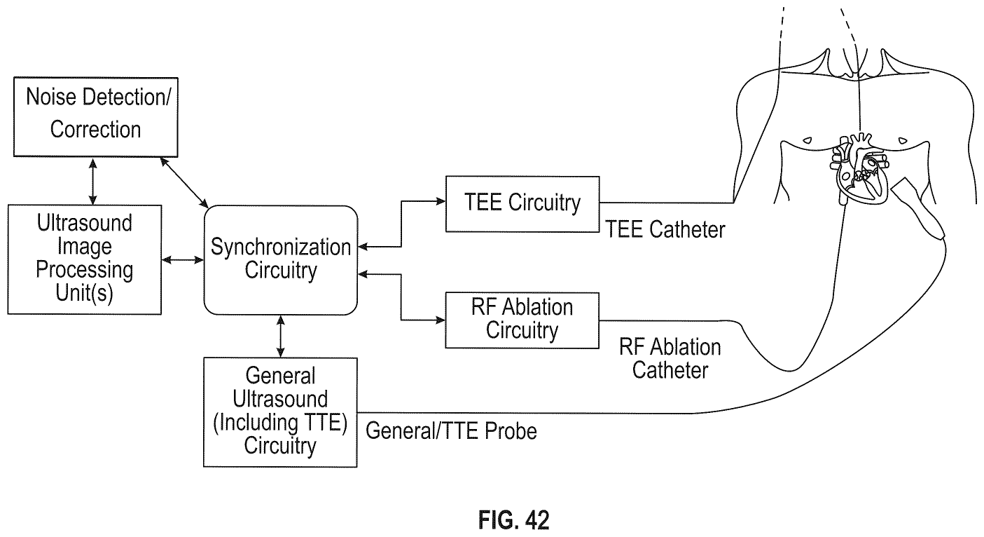

Except for detecting and correcting noise for the system involving three ultrasound modalities and RF ablation modality shown in FIG. 34, the noise detection/correction system 800 can be easily implemented in a reduced multiple-modality system involving one ultrasound modality and an RF ablation modality, or involving two ultrasound modalities and an RF ablation modality. These reduced systems include the ones shown in FIG. 37 for ICE catheter and RF ablation catheter, in FIG. 38 for TEE catheter and RF ablation catheter, in FIG. 39 for general/TTE probe and RF ablation catheter, in FIG. 40 for ICE and TEE catheters and RF ablation catheter, in FIG. 31 for ICE catheter, general and TTE probe and RF ablation catheter, and in FIG. 42 for TEE catheter, general/TTE probe and RF ablation catheter.

In fact, the noise detection/correction system 800 any of the multiple-modality systems described previously when the synchronization circuitry described previously in application is implemented on the system. More broadly it can be implemented with the synchronization circuitry in any multiple-modality system supporting any two or more of the following modalities: the ICE catheter, the IVUS catheter, the RF ablation catheter, the FFR interface catheter, the TEE catheter, and/or the general/TEE probe.

Explicitly for the modalities listed above, the noise detection/correction system together with the synchronization circuitry can be implemented in a multiple modality system supporting an ICE catheter in addition to any combination of one or more of an IVUS catheter, an RF ablation catheter, an FFR interface catheter, a TEE catheter, and/or a general/TTE probe. And the noise detection/correction system together with the synchronization circuitry can be implemented in a multiple modality system supporting an IVUS catheter in addition to any combination of one or more of an RF ablation catheter, an FFR interface catheter, a TEE catheter, and/or a general/TTE probe. Further, the noise detection/correction system together with the synchronization circuitry can be implemented in a multiple modality system supporting an FR ablation catheter in addition to any combination of any one or more or an FFR interface catheter, a TEE catheter, and/or a general/TTE probe. In addition to that, the noise detection/correction system together with the synchronization circuitry can be implemented in a multiple modality system supporting an FFR interference catheter in addition to a TEE catheter and/or a general/TTE probe. To exhaust the list, the noise detection/correction system together with the synchronization circuitry can be implemented in a multiple-modality system supporting a TEE catheter and/or a general/TTE probe.

### EXAMPLE EMBODIMENTS

In a first embodiment, a multiple-modality system disposed in a housing comprises a battery module; a wireless module, the wireless module capable of transmitting wireless signals to communicate with peripheral equipment; a computing module, the computing module having electronics and software loaded thereon capable of processing data; and a modality circuitry module, the modality circuitry module capable of supporting a plurality of modalities. The modality circuitry module is capable of supporting an intracardiac echocardiography (ICE) catheter modality and an intravascular ultrasound (IVUS) catheter modality. The modality circuitry module is capable of supporting an intracardiac echocardiography (ICE) catheter modality, an intravascular ultrasound (IVUS) catheter modality, and a radiofrequency (RF) ablation catheter modality. The modality circuitry module is capable of supporting an intracardiac echocardiography (ICE) catheter modality, an intravascular ultrasound (IVUS) catheter modality, a radiofrequency (RF) ablation catheter modality, and a fractional flow reserve (FFR) catheter modality. The modality circuitry module is capable of supporting an intracardiac echocardiography (ICE) catheter modality, a transthoracic echocardiography (TTE) probe modality, transesophageal echocardiography (TEE) catheter modality. The modality circuitry module is capable of supporting modalities selected from the group of modalities consisting of an intracardiac echocardiography (ICE) catheter modality, a transthoracic echocardiography (TTE) probe modality, a transesophageal echocardiography (TEE) catheter modality, a radiofrequency (RF) ablation catheter modality. The housing is a chassis with dimensions equal to or smaller than 15 inch×12 inch×6 inch. The dimensions of the chassis is equal to or smaller than 12 inch×10 inch×4 inch. The housing is a reusable catheter handle. The reusable catheter handle is a universal catheter handle capable of supporting a plurality of catheter modalities. The battery module includes a rechargeable battery set. When fully charged the rechargeable battery set is capable of suppling power to operate the multiple-modality system for more than 4 hours.

In a second embodiment, a synchronization system capable of reducing interference for a plurality of modality systems, comprises a scheduler, the scheduler capable of sending out signals to cause the plurality of modalities to operate sequentially forming repetitive operation cycles with a cycling frequency, wherein at a specific time no more than one modality is running; and a controller, the controller starting and stopping each of the plurality of modalities according to signals received from the scheduler. The controller includes a clock synchronizer, the clock synchronizer capable of generating a clock pulse signal with a clock pulse frequency, wherein the clock pulse frequency is substantially higher than the cycling frequency; a clock-gating controller, the clock-gating controller capable of starting, pausing, and stopping the clocking for each of the plurality of modalities according to the signals from the scheduler; a power-gating controller, the power-gating controller capable of starting, pausing, and stopping the power for each of the plurality of modalities according to the signals from the scheduler; and wherein each of the plurality of modalities is in operation mode only when the clocking is started by the clock-gating controller for the modality and the power is started by the power-gating controller. Each of the plurality of modality systems includes an intracardiac echocardiography (ICE) catheter modality and an intravascular ultrasound (IVUS) catheter modality. Each of the plurality of modality systems includes an intracardiac echocardiography (ICE) catheter modality, an intravascular ultrasound (IVUS) catheter modality, and a radiofrequency (RF) ablation catheter modality. Each of the plurality of modality systems includes an intracardiac echocardiography (ICE) catheter modality, an intravascular ultrasound (IVUS) catheter modality, a radiofrequency (RF) ablation catheter modality, and a fractional flow reserve (FFR) catheter modality. Each of the plurality of modality systems includes an intracardiac echocardiography (ICE) catheter modality, a transthoracic echocardiography (TTE) probe modality, and transesophageal echocardiography (TEE) catheter modality. Each of the plurality of modality systems includes modalities selected from the group consisting of an intracardiac echocardiography (ICE) catheter modality, a transthoracic echocardiography (TTE) probe modality, a transesophageal echocardiography (TEE) catheter modality, and a radiofrequency (RF) ablation catheter modality.

In a third embodiment, a method for synchronizing a plurality of modality systems for optimal performance for each of the modality systems, comprises the steps of: establishing a scheduler, the scheduler capable of sending out signals to cause the plurality of modalities to operate sequentially forming repetitive operation cycles with a cycling frequency, wherein at a specific time no more than one modality is running; and establishing a controller, the controller starting and stopping each of the plurality of modalities according to signals received from the scheduler. The step of establishing a controller comprises the steps of: generating a clock pulse signal with a clock pulse frequency, wherein the clock pulse frequency is substantially higher than the cycling frequency; establishing a clock-gating controller, the clock-gating controller capable of starting, pausing, and stopping the clocking for each of the plurality of modalities according to the signals from the scheduler; establishing a power-gating controller, the power-gating controller capable of starting, pausing, and stopping the power for each of the plurality of modalities according to the signals from the scheduler; and wherein each of the plurality of modalities is in operation mode only when the clocking is started by the clock-gating controller for the modality and the power is started by the power-gating controller. Each of the plurality of modality systems includes an intracardiac echocardiography (ICE) catheter modality and an intravascular ultrasound (IVUS) catheter modality. Each of the plurality of modality systems includes an intracardiac echocardiography (ICE) catheter modality, an intravascular ultrasound (IVUS) catheter modality, and a radiofrequency (RF) ablation catheter modality. Each of the plurality of modality systems includes an intracardiac echocardiography (ICE) catheter modality, an intravascular ultrasound (IVUS) catheter modality, a radiofrequency (RF) ablation catheter modality, and a fractional flow reserve (FFR) catheter modality. Each of the plurality of modality systems includes an intracardiac echocardiography (ICE) catheter modality, a transthoracic echocardiography (TTE) probe modality, and a transesophageal echocardiography (TEE) catheter modality. Each of the plurality of modality systems includes modalities selected from the group consisting of an intracardiac echocardiography (ICE) catheter modality, a transthoracic echocardiography (TTE) probe modality, a transesophageal echocardiography (TEE) catheter modality, and a radiofrequency (RF) ablation catheter modality.

In a fourth embodiment, a method for detecting and ablating lesion using a multiple-modality system including an intracardiac echocardiography (ICE) catheter and a radiofrequency (RF) ablation catheter, comprising the steps of: generating an ultrasound image frame with the ICE catheter; determining if lesion is detected using a lesion detection convolutional neural network (CNN); if lesion is detected, optimizing ablation power and pulse for the RF ablation catheter and proceeding to ablate the lesion; and if lesion is not detected, going back to the beginning of the loop to generate another ultrasound image frame with the ICE catheter. The method further comprises the steps of: if lesion is detected, determining if deep-learning guided lesion imaging is needed; if the answer yes, rotating the ICE catheter tip using servo control and proceeding to determine if full lesion ablation is acquired; and if the answer is yes, going to the step of optimizing ablation power and pulse for the RF ablation catheter and proceeding to ablate the lesion.

In a fifth embodiment, a method for detecting and correcting noise in a multiple-modality system including an echocardiography modality for optimal ultrasound image quality, comprises the steps of: generating an ultrasound image frame using the echocardiography modality; detecting noise using a noise detector; determining if abnormal ultrasound noise is detected; if noise is detected, proceeding to adjust ultrasound control parameters and go back to the beginning of the loop to generate an ultrasound image frame; and if noise is not detected, proceeding to noise frequency analysis and auto filter generation for denoising and go back to generate an ultrasound image frame. The echocardiography modality is an ICE catheter. The echocardiography modality is a TEE catheter. The echocardiography modality is a TTE/general probe.

In a sixth embodiment, a multiple-modality system disposed in a housing. comprises: a battery module; a communication module, the communication module capable of transmitting signals to communicate with peripheral equipment; a computing module, the computing module having electronics and software loaded thereon capable of processing data; and a modality circuitry module, the modality circuitry module capable of supporting a plurality of modalities. The modality circuitry module is capable of supporting an intracardiac echocardiography (ICE) catheter modality and an intravascular ultrasound (IVUS) catheter modality. The modality circuitry module is capable of supporting an intracardiac echocardiography (ICE) catheter modality, an intravascular ultrasound (IVUS) catheter modality, and a radiofrequency (RF) ablation catheter modality. The modality circuitry module is capable of supporting an intracardiac echocardiography (ICE) catheter modality, an intravascular ultrasound (IVUS) catheter modality, a radiofrequency (RF) ablation catheter modality, and a fractional flow reserve (FFR) catheter modality. The modality circuitry module is capable of supporting an intracardiac echocardiography (ICE) catheter modality, a transthoracic echocardiography (TTE) probe modality, and a transesophageal echocardiography (TEE) catheter modality. The modality circuitry module is capable of supporting modalities selected from the group of modalities consisting of an intracardiac echocardiography (ICE) catheter modality, a transthoracic echocardiography (TTE) probe modality, a transesophageal echocardiography (TEE) catheter modality, a radiofrequency (RF) ablation catheter modality. The housing is a chassis with dimensions equal to or smaller than 15 inch×12 inch×6 inch. The dimensions of the chassis is equal to or smaller than 12 inch×10 inch×4 inch. The housing is a reusable catheter handle. The reusable catheter handle is a universal catheter handle capable of supporting a plurality of catheter modalities. The battery module includes a rechargeable battery set. When fully charged the rechargeable battery set is capable of suppling power to operate the multiple-modality system for more than 4 hours.

### Implementation Consideration

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

It will also be understood that, when a feature or element (for example, a structural feature or element) is referred to as being “connected”, “attached” or “coupled” to another feature or element, it may be directly connected, attached or coupled to the other feature or clement or intervening features or elements may be present. In contrast, when a feature or element is referred to as being “directly connected”, “directly attached” or “directly coupled” to another feature or element, there may be no intervening features or elements present. Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments and implementations only and is not intended to be limiting. For example, as used herein, the singular forms “a”, “an” and “the” may be intended to include the plural forms as well, unless the context clearly indicates otherwise. It will be further understood that the terms “comprises” and/or “comprising,” when used in this specification, specify the presence of stated features, steps, operations, processes, functions, elements, and/or components, but do not preclude the presence or addition of one or more other features, steps, operations, processes, functions, elements, components, and/or groups thereof. As used herein, the term “and/or” includes any and all combinations of one or more of the associated listed items and may be abbreviated as “/”.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Spatially relative terms, such as “forward,” “rearward,” “under,” “below,” “lower,” “over,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features due to the inverted state. Thus, the term “under” may encompass both an orientation of over and under, depending on the point of reference or orientation. The device may be otherwise oriented (rotated 90 degrees or at other orientations) and the spatially relative descriptors used herein interpreted accordingly. Similarly, the terms “upwardly”, “downwardly”, “vertical”, “horizontal” and the like may be used herein for the purpose of explanation only unless specifically indicated otherwise.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

Although various illustrative embodiments have been disclosed, any of a number of changes may be made to various embodiments without departing from the teachings herein. For example, the order in which various described method steps are performed may be changed or reconfigured in different or alternative embodiments, and in other embodiments one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

The examples and illustrations included herein show, by way of illustration and not of limitation, specific embodiments in which the disclosed subject matter may be practiced. As mentioned, other embodiments may be utilized and derived therefrom, such that structural and logical substitutions and changes may be made without departing from the scope of this disclosure. Such embodiments of the disclosed subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve an intended, practical or disclosed purpose, whether explicitly stated or implied, may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The disclosed subject matter has been provided here with reference to one or more features or embodiments. Those skilled in the art will recognize and appreciate that, despite of the detailed nature of the example embodiments provided here, changes and modifications may be applied to said embodiments without limiting or departing from the generally intended scope. These and various other adaptations and combinations of the embodiments provided here are within the scope of the disclosed subject matter as defined by the disclosed elements and features and their full set of equivalents.

What is claimed is:

1. A method for detecting and correcting noise in a multiple-modality system including an echocardiography modality for optimal ultrasound image quality, the method comprising:
generating an ultrasound image frame using the echocardiography modality;
detecting noise using a noise detector;
determining if abnormal ultrasound noise is detected;
if noise is detected, proceeding to adjust ultrasound control parameters and generating a subsequent ultrasound image frame; and
if noise is not detected, proceeding to noise frequency analysis and auto filter generation for denoising and generating a subsequent ultrasound image frame.

2. The method of claim 1, further comprising if noise is detected, proceeding to adjust radiofrequency (RF) ablation control parameters.

3. The method of claim 1, wherein the echocardiography modality is an intracardiac echocardiography (ICE) catheter.

4. The method of claim 1, wherein the echocardiography modality is a transesophageal echocardiography (TEE) catheter.

5. The method of claim 1, wherein the echocardiography modality is a transthoracic echocardiography (TTE).

6. The method of claim 1, wherein the echocardiography modality is a radiofrequency (RF) ablation catheter.

7. A method for detecting and ablating lesion using a multiple-modality system including an intracardiac echocardiography (ICE) catheter and a radiofrequency (RF) ablation catheter, the method comprising:
generating an ultrasound image frame with the ICE catheter;
determining if lesion is detected using a lesion detection convolutional neural network (CNN);
if lesion is detected, optimizing ablation power and pulse for the RF ablation catheter and proceeding to ablate the lesion; and
if lesion is not detected, generating another ultrasound image frame with the ICE catheter.

8. The method of claim 7, further comprising:
if lesion is detected, determining if deep-learning guided lesion imaging is needed;
if deep-learning guided lesion imaging is needed, rotating a tip of the ICE catheter using servo control and proceeding to determine if full lesion ablation is acquired; and
if full lesion ablation is acquired, optimizing ablation power and pulse for the radiofrequency (RF) ablation catheter and proceeding to ablate the lesion.

9. The method of claim 7, wherein the multiple-modality system further comprises a transesophageal echocardiography (TEE) catheter.

10. The method of claim 7, wherein the multiple-modality system further includes a transthoracic echocardiography (TTE).

11. The method of claim 7, further comprising generating an ultrasound image frame with a transesophageal echocardiography (TEE) catheter and a transthoracic echocardiography (TTE) catheter.

12. The method of claim 11, further comprising detecting noise and determining if abnormal ultrasound noise is detected.

13. The method of claim 12, wherein if abnormal ultrasound noise is detected, adjusting ultrasound control parameters and radiofrequency (RF) control parameters.

14. The method of claim 12, wherein if abnormal ultrasound noise is not detected, analyzing noise frequency and auto filter generation.

15. A noise detection and correction system comprising:
a plurality of modalities each configured to generate an ultrasound image frame; and
a noise detector in electrical communication with the plurality of modalities;
wherein the noise detector is configured to:
receive the ultrasound image frame;
detect noise in the ultrasound image frame; and
determine if abnormal ultrasound noise.

16. The noise detection and correction system of claim 15, wherein the plurality of modalities comprises an intracardiac echocardiography (ICE) catheter.

17. The noise detection and correction system of claim 15, wherein the plurality of modalities comprises a transesophageal echocardiography (TEE) catheter.

18. The noise detection and correction system of claim 15, wherein the plurality of modalities comprises a transthoracic echocardiography (TTE).

19. The noise detection and correction system of claim 15, wherein the plurality of modalities comprises an intracardiac echocardiography (ICE) catheter, a transesophageal echocardiography (TEE) catheter, and a transthoracic echocardiography (TTE).

20. The noise detection and correction system of claim 15, further comprising a radiofrequency (RF) ablation controller in electrical communication with the noise detector.
